(19) United States
(12) Patent Application Publication
(10) Pub. No.: US 2025/0114419 A1
(43) Pub. Date: Apr. 10, 2025

(54) INTESTINAL HEALTH PROMOTING COMPOSITIONS

(71) Applicant: NSE Products, Inc., Provo, UT (US)

(72) Inventors: Angela Mastaloudis, Holladay, UT (US); Steven M. Wood, Santaquin, UT (US); Shelly Hester, Draper, UT (US); Mark Bartlett, Orem, UT (US)

(21) Appl. No.: 18/801,755

(22) Filed: Aug. 12, 2024

Related U.S. Application Data
(63) Continuation of application No. 16/346,100, filed on Apr. 29, 2019, now abandoned.

(51) Int. Cl. A61K 36/45; A61K 31/352; A61K 31/702; A61K 31/733; A61K 36/185; A61K 36/73; A61K 36/899; A61P 3/04; A61P 29/00

(52) U.S. Cl. CPC A61K 36/45; A23L 33/105; A23P 10/40; A61K 9/14; A61K 31/352; A61K 31/702; A61K 31/733; A61K 36/185; A61K 36/899; A61K 45/06; A61P 1/00; A23V 2002/00; A23V 2200/3202; A23V 2250/2104; A23V 2250/2106; A23V 2250/284; A23V 2250/5062

(57) ABSTRACT

Compositions for promoting intestinal health are disclosed and described. In one example, the composition can include a combination of cyanidins and delphinidins, in an amount sufficient to treat intestinal hyperpermeability. In a further example, the composition can further comprise a prebiotic blend and fructooligosaccharides. Further presented herein, is a method of treating a condition or disorder related to gastrointestinal health in a subject. In one example, the method can include maximizing tight junction integrity in epithelial cells of gastrointestinal tract of the subject.

### PRIORITY DATA

This application is a continuation of U.S. patent application Ser. No. 16/346,100, filed Apr. 29, 2019, which is a 371 U.S. Nationalization of Patent Cooperation Treaty Application PCT/US16/59226, filed on Oct. 27, 2016, each of which is incorporated herein by reference in their entirety.

### GOVERNMENT INTEREST

A portion of the research for this invention was made with government support under a grant awarded by the National Institute of Food and Agriculture. The government has certain rights in the invention.

### BACKGROUND

The gastrointestinal system is a complex network of tissues, organs, host cells, and bacterial cells. Aging, diets, medications, disruptions in the intestinal microbiota, and an individual's lifestyle conditions can negatively affect this network. These effects can include disrupting the balance of the gut microbiota, increased inflammation in the gastrointestinal tissues, and a disruption in the epithelial cell barrier of the gastrointestinal system. These effects can ultimately lead to systemic issues. Formulation and methods that promote and restore intestinal health would provide a benefit.

### BRIEF DESCRIPTION OF THE DRAWINGS

Features and advantages of the invention will be apparent from the detailed description that follows, and which taken in conjunction with the accompanying figures together illustrate features of the invention. It is understood that the figures merely depict exemplary embodiments and results and are therefore, not to be considered limiting in scope.

FIG. 1 schematically displays the human gastrointestinal tract and related structures;

FIG. 2 schematically displays the small intestine and the epithelial cell layer lining the intestines;

FIG. 3 schematically displays an overview of the tight junctions in epithelial cells and their systemic impact when the tight junction barrier is disrupted;

FIG. 4 schematically displays the intestinal epithelial cell layer, tight junctions, the paracellular pathway and the transcellular pathway;

FIG. 5 schematically displays the chemical structure of cyanidin;

FIG. 6 schematically displays the chemical structure of delphinidin;

FIG. 7 schematically displays the chemical structure of petunidin;

FIG. 8 schematically displays the chemical structure of peonidin;

FIG. 9 schematically displays the chemical structure of malvidin;

FIG. 10 graphically displays the transepithelial electrical resistance (TEER) method in accordance with one example of the disclosure;

FIG. 11 graphically displays the TEER resistance for various extracts in accordance with one example of the disclosure;

FIG. 12 graphically displays paracellular transport for various extracts in accordance with one example of the disclosure;

FIG. 13 graphically displays TEER resistance for various extracts in accordance with one example of the disclosure;

FIG. 14 graphically displays PCP FITC-dextran for various extracts in accordance with one example of the disclosure;

FIG. 15 graphically displays the TEER resistance for cyanidins in accordance with one example of the disclosure;

FIG. 16 graphically displays the TEER resistance for delphinidin in accordance with one example of the disclosure;

FIGS. 17 & 18 graphically display TEER resistance for epicatechin in accordance with one example of the disclosure;

FIG. 19 graphically displays TEER resistance for catechin in accordance with one example of the disclosure;

FIG. 20 graphically displays TEER resistance for total catechins in accordance with one example of the disclosure;

FIG. 21 graphically displays average colon length of mice on different diets in accordance with one example presented herein;

FIG. 22 graphically displays average colon weight of mice on different diets in accordance with one example presented herein;

FIG. 23 graphically displays average colon weight/length of mice on different diets in accordance with one example presented herein;

FIG. 24 graphically displays body weight gain and FITC dextran permeability of mice on different diets in accordance with on example presented herein;

FIG. 25 graphically displays the FITC-DX paracellular transport of mice on different diets in accordance with one example presented herein;

FIG. 26 graphically displays endotoxin levels measured of mice on different diets in accordance with one example presented herein;

FIG. 27 graphically displays GTT (AUC) levels measured of mice on different diets in accordance with one example presented herein;

FIG. 28 graphically displays ITT (AUC) levels measured of mice on different diets in accordance with one example presented herein;

FIG. 29 graphically displays endotoxin and glucose tolerance test levels measured in accordance with one example present herein;

FIG. 30 graphically displays endotoxin and fasting insulin levels measured in accordance with one example presented herein;

FIG. 31 graphically displays endotoxin and IL-6 test levels measured in accordance with one example present herein;

FIG. 32 graphically displays endotoxin and IL-1β levels measured in accordance with one example presented herein;

FIG. 33 graphically displays endotoxin and IL-1α test levels measured in accordance with one example present herein;

FIG. 34 graphically displays HOMA-IR levels measured of mice on different diets in accordance with one example presented herein;

FIG. 35 graphically displays adiponectin levels measured of mice on different diets in accordance with one example presented herein;

FIG. 36 graphically displays leptin levels measured of mice on different diets in accordance with one example presented herein;

FIGS. 37 & 38 graphically displays triglyceride levels measured of mice on different diets in accordance with one example presented herein;

FIGS. 39 & 40 graphically displays cholesterol levels measured of mice on different diets in accordance with one example presented herein;

FIG. 41 graphically displays liver triglyceride levels measured of mice on different diets in accordance with one example presented herein;

FIG. 42 photographically displays mice livers extracted from mice fed different diets prior to be euthanized in accordance with one example presented herein;

FIG. 43 photographically displays mice droppings from mice on different diets in accordance with one example presented herein;

FIG. 44 graphically displays the average firmicutes:bacterodetes ratio of the gut microbiome following supplementation with a composition as presented herein;

FIG. 45 graphically displays calprotectin concentrations following supplementation with a composition as presented herein in;

FIG. 46 graphically displays a change in baseline BSS scores and bowel movements following supplementation with a composition as presented herein; and

FIG. 47 graphically displays the change in baseline scores for bloating, abdominal pain, and gas following supplementation with a composition as presented herein.

### DETAILED DESCRIPTION

Before invention embodiments are disclosed and described, it is to be understood that no limitation to the particular structures, process steps, or materials disclosed herein is intended, but also includes equivalents thereof as would be recognized by those ordinarily skilled in the relevant arts. It should also be understood that terminology employed herein is used to describe particular examples only and is not intended to be limiting. Unless defined otherwise, all technical and scientific terms used herein have the same meaning as commonly understood by one of ordinary skill in the art to which this disclosure belongs. In the specification and the appended claims, the singular forms include plural referents unless the context clearly dictates otherwise.

As used herein, the singular forms “a,” “an,” and “the” specifically provide express support for plural referents, unless the content clearly dictates otherwise. For example, “a prebiotic fiber” provides support for one or more prebiotic fibers.

The term “about” is used herein refers to a degree of deviation. It means approximately, in the region of, roughly, or around. When the term “about” is used in conjunction with a numerical range, it modifies that range by extending the boundaries slightly above and slightly below the numerical values set forth. It is understood that support in this specification for numerical values used in connection with the term “about” is also provided for the exact numerical value itself as though “about” were not used.

Concentrations, amounts, and other numerical data may be expressed or presented herein in a range format. It is to be understood that such a range format is used merely for convenience and brevity and thus should be interpreted flexibly to include not only the numerical values explicitly recited as the limits or endpoints of the range, but also to include all the individual numerical values and/or sub-ranges encompassed within that range as if each numerical value (including fractions) and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range for example, are individual values such as 2, 2.6, 3, 3.8, and 4 and sub-ranges such as from 1-3, from 2-4, and from 3-5, etc., as well as 1, 2, 3, 4, and 5, individually.

As used herein a “concentrate” refers to an extract of a source that contains at least the same amount of active fractions, compounds, or other constituents, in a smaller volume than in the source itself. In one example, a “concentrate” may be a dried powder derived from a component that does not include the use of any solvents during the concentration process.

Comparative terms such as “more effectively,” “greater than,” “improved,” “enhanced,” and like terms can be used to state a result achieved or property present in a formulation or process that has a measurably better or more positive outcome than the thing to which comparison is made. In some instances comparison may be made to the prior art or to the status of a property before administration of a formulation or method that results in the more positive outcome.

As used herein, “comprises,” “comprising,” “containing,” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term in the written description, like “comprising” or “including,” it is understood that direct support should be afforded also to “consisting essentially of” language, as well as, “consisting of” language as if stated explicitly and vice versa.

The term “dosage unit” is understood to mean a single unit of composition which is capable of being administered to a subject or patient, to provide an amount of an active agent sufficient to achieve, or contribute to, a therapeutic effect to be achieved. In some embodiments, a “dosage unit” is a unit and that may be readily handled and packed, remaining as a physically and chemically stable unit dose comprising either the active ingredient as such or a mixture of it with solid or liquid pharmaceutical vehicle materials. Moreover, “dosage” and “dose” can refer to such a dosage unit. Alternatively, a “dosage” or “dose” can encompass multiple dosage units which collectively provide a desired amount of active agent to be administered to a subject at a singular point in time. Multiple doses or dosages can be utilized according to a schedule in order to establish a dosing regimen.

The phrase “effective amount,” “therapeutically effective amount,” or “therapeutically effective rate(s)” of an active ingredient refers to a non-toxic, but sufficient amount or delivery rates of the active ingredient, to achieve therapeutic results in treating a disease or condition for which the active agent is being delivered. It is understood that various biological factors may affect the ability of a substance to perform its intended task. Therefore, an “effective amount,” “therapeutically effective amount,” or “therapeutically effective rate(s)” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

The term “extract” refers to those substances prepared using a solvent, e.g., ethanol, water, steam, superheated water, methanol, hexane, chloroform liquid, liquid CO₂, liquid N₂, propane, supercritical CO₂, or any combination thereof. Extracts, as used herein, can refer to an extract in a liquid form, or can refer to a product obtained from further processing of the liquid form, such as a dried powder or other solid form. Extracts may take many forms including but not limited to: solid, liquid, particulate, chopped, distillate, etc. and may be performed by any number of procedures or protocols, such as chopping, grinding, pulverizing, boiling, steaming, soaking, steeping, infusing, applying a gas, etc., and may employ any suitable reagents, such as water, alcohol, steam, or other organic materials. Extracts typically have a given purity percentage and can be relatively to highly pure. In some embodiments, extracts can be phytoextracts made from specific parts of a source, such as the skin, pulp, leaves, flowers, fruits, kernels, seeds, of a plant etc., or can be made from the whole source. In some aspects, an extract may include one or more active fractions or active agents. In some aspects, the purity of an extract can be controlled by, or be a function of the extraction process or protocol.

As used herein, “formulation” and “composition” can be used interchangeably and refer to a combination of at least two ingredients. In some embodiments, at least one ingredient may be an active agent or otherwise have properties that exert physiologic activity when administered to a subject.

Formulation or compositional ingredients included or recited herein are to be presumed to be in wt % unless specifically stated otherwise. In addition, ingredient amounts presented in the form of ratios are to be presumed to be in wt % (e.g. % w/w) ratios.

The phrase, “intestinal hypermeability” refers to a higher than normal (i.e. higher than that of an average subject) permeability of the gastrointestinal system and can in some instances refer to increased permeability in the stomach, small intestines and/or large intestines.

As used herein, “linear inhibitory effect” or “dose-response” refers to a linear decrease in secretion or biosynthesis resulting from all concentrations of the inhibiting material over a dose-response curve. For example, inhibition at low concentrations followed by a failure of inhibition or increased secretion at higher concentrations represents a lack of a linear inhibitory effect.

The term “or” is used in the “inclusive” sense of “and/or” and not the “exclusive” sense of “either/or.”

As used herein, “pharmaceutically acceptable” refers generally to materials, which are suitable for administration to a subject in connection with an active agent or ingredient. For example, a “pharmaceutically acceptable carrier” can be any substance or material that can be suitably combined with an active agent to provide a composition or formulation suitable for administration to a subject. Excipients, diluents, and other ingredients used in or used to prepare a formulation or composition for administration to a subject can be used with such term.

The term “prevent” and its variants refer to prophylaxis against a particular undesirable physiological condition. The prophylaxis may be partial or complete. Partial prophylaxis may result in the delayed onset of a physiological condition. An individual skilled in the art will recognize the desirability of delaying onset of a physiological condition, and will know to administer the compositions of the invention to subjects who are at risk for certain physiological conditions in order to delay the onset of those conditions. For example, the person skilled in the art will recognize that obese subjects are at elevated risk for coronary artery disease. Thus, the person skilled in the art will administer compositions of the invention in order to improve the gut microbiota in an obese individual.

As used herein, a “subject” refers to an individual receiving treatment. In one aspect, a subject can be a mammal. In another aspect, a subject can be a human. In another aspect, the subject can be a domesticated animal or livestock.

As used herein, “substantial,” or “substantially,” when used in reference to a quantity or amount of a material, an effect, or a specific characteristic of a composition thereof, refers to an amount that is sufficient to provide an effect that the material or a characteristic the material was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition, or are included only in amounts which are small enough so as to have no measurable effect on the composition.

As used herein, the term “insignificant” or “clinically insignificant” refers to the extent of an effect of administering a composition to a subject. For example, if the extent of the result does not cause a clinical change in the subject, then the result is clinically insignificant.

The terms “treat,” “treating,” or “treatment” as used herein and as well understood in the art, mean an approach for obtaining beneficial or desired results, including without limitation clinical results in a subject being treated. Beneficial or desired results can include, but are not limited to, alleviation or amelioration of one or more signs or symptoms of a condition, diminishment of extent of disease, stabilizing (i.e. not worsening) the state of a disease or condition, delaying or slowing of disease progression, amelioration or palliation of the disease state, diminishment of the reoccurrence of disease, and remission (whether partial or total), whether detectable or undetectable. “Treat,” “treating” and “treatment” can also mean prolonging survival as compared to expected survival if not receiving treatment and can be prophylactic. Such prophylactic treatment can also be referred to as prevention or prophylaxis of a disease or condition. The prophylaxis may be partial or complete. Partial prophylaxis may result in the delayed onset of a physiological condition.

As used herein, the term “solvent” refers to a liquid of gaseous, aqueous, or organic nature possessing the necessary characteristics to extract solid material from a plant product. Examples of solvents would include, but not limited to, water, steam, superheated water, methanol, ethanol, ethyl acetate, hexane, chloroform, liquid CO₂, liquid N₂, propane, or any combinations of such materials.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Any steps recited in any method or process claims may be executed in any order and are not limited to the order presented in the claims unless otherwise stated.

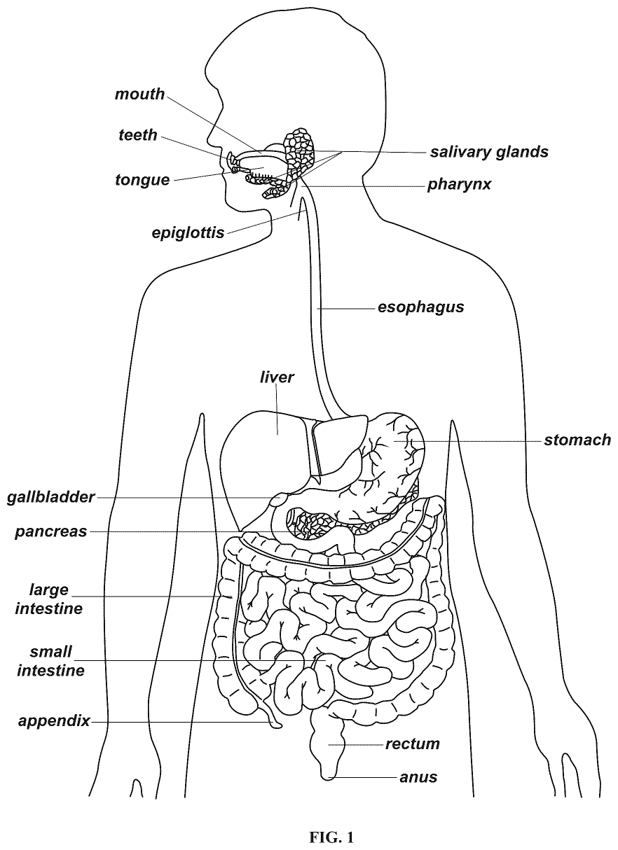

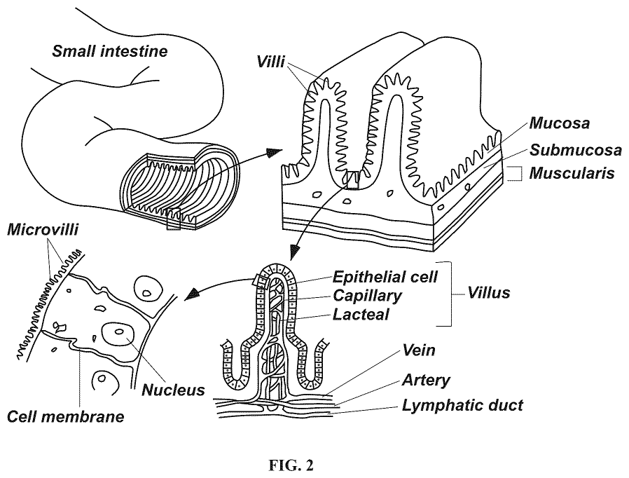

The gastrointestinal system is a tract of connected and related structures that are involved in the digestion of food and absorption of energy and nutrients. The gastrointestinal tract, shown in FIG. 1, consists of all the structures between the mouth and the anus. The whole gastrointestinal tract is about nine meters long and can be divided into an upper gastrointestinal tract and a lower gastrointestinal tract. The lower gastrointestinal tract includes the small intestine and the large intestine. The small intestine is about 20 feet in length and has a highly folded structure that includes finger like protections called villi. Within the villi is a single layer of epithelial cells and a layer of capillaries. The main function of the small intestine is to absorb products of digestion. Nutrients can pass through the layer of epithelial cells and enter the capillaries underneath. These nutrients can eventually enter larger blood vessels and travel to the liver where they are processed and regulated for release into the body. The large intestine is about 3 feet long and is used to collect the solid material that was not digested in the small intestine and to absorb water. The large intestine is loaded with bacteria that synthesize vitamins.

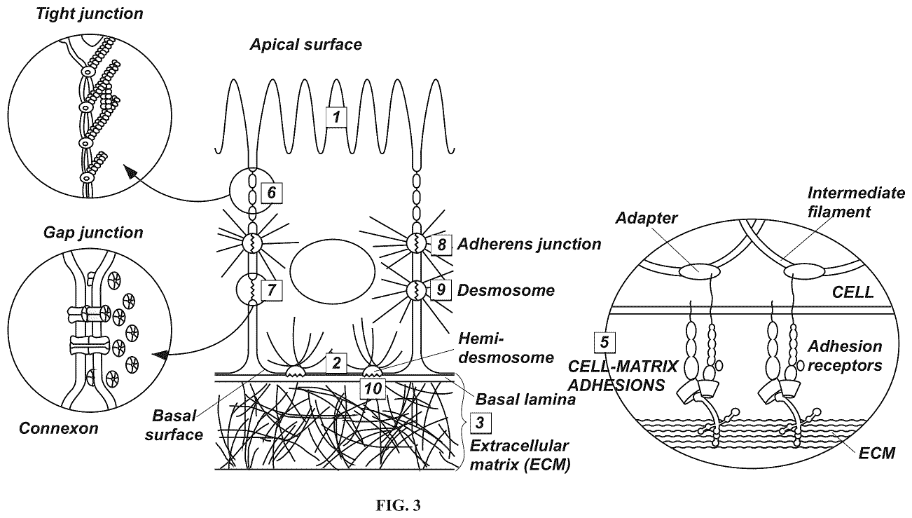

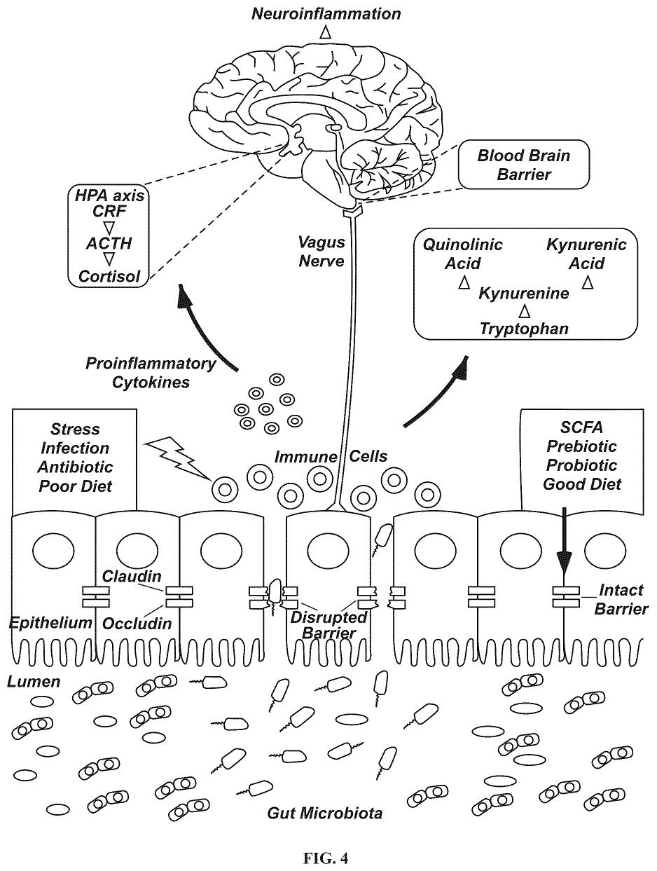

As previously mentioned, the small intestine has a barrier composed of a single layer of epithelial cells. These epithelial cells are sealed by tight junctions. The tight junctions can modulate intestinal permeability by regulating paracellular transport of water and ions and the transcellular pathway. See, FIG. 3. In addition to being responsible for absorption of nutrients, this cell layer also plays a role in maintaining the mucosal immune homeostasis, preventing inflammation, and constitutes the first line of defense against the entry of noxious bacteria/bacterial toxins and/or other antigens that can initiate chronic inflammation. Any bacteria, bacterial toxins, antigens, water, and ions that pass through this cell layer can enter the blood stream, affect other organs, and can have systemic effects on the individual overall. See, FIG. 4. Lifestyle and dietary factors such as high-intensity exercise, high-fat diets, and over nutrition can also influence intestinal permeability and can play a role in increasing toxin permeability across tight junctions and into circulation.

Disruption of the tight junction results in a leaky tight junction barrier and can lead to an increase in intestinal permeability. Increased permeability can be a major factor in the pathophysiology of several inflammation and obesity related pathologies. Low levels of chronic inflammation can negatively affect intestinal barrier permeabilization.

Tumor necrosis factor alpha (TNFα) can also be a central underlying mediator. TNFα triggers apoptosis; however, these changes in distribution occur through its capacity to promote barrier permeabilization and expression of select tight junction proteins. Ultimately, TNFα can play a role in promoting tight junction barrier dysfunction. Loss of tight junction functionality and increased intestinal permeability can be contributors to the pathology of allergies (e.g. celiac disease), inflammatory bowel diseases (Crohn's disease and ulcerative colitis), food intolerances, dyspepsia, low levels of chronic intestinal inflammation (e.g. those associated with obesity and type I and II diabetes), insulin resistance, autism, multiple sclerosis, malnutrition, metabolic syndrome, cancer, asthma, atopy, and rheumatoid arthritis.

Another factor in the health of an individual's gastrointestinal system is the microbiota of the gut. The microbiota is a complex network of bacteria, archae, viruses, and eukarya that influence an individual's health and physiology. The gastrointestinal microbiota has been estimated to reach approximately 3.9×10¹³ microorganisms. The composition of an individual's microbiota can vary greatly in numbers, diversity, histology, and activities. The richness and diversity of the gut microbiota has been shown to be significantly influenced by antibiotics, age, diet, ethnicity, geographical location, physiological stress, psychological stress, and sex.

Alterations in the gut microbiota can cause an increase in endotoxin production. A microbiota that has few and less diverse beneficial bacteria and has greater numbers and more diverse non-beneficial bacteria can occur with age and/or conditions associated with accelerated aging, such as, obesity or a high fat diet. An imbalance of the microbiota can also lead to increased inflammation of the gastrointestinal lining, changes in the integrity of the intestinal cell wall, and can led to gut permeability. These changes can contribute to the incidence of gastrointestinal infections, asthma/atopy, obesity, metabolic syndrome, cancer, rheumatoid arthritis, Crohn's disease, and ulcerative colitis. In contrast, a balanced and diverse microbiota can provide resistance to infections, allow for healthy aging, prevent intestinal disorders, contribute to polyphenol metabolism, and can generate absorbable bioactives. The gut microbiome can also play a role in metabolism, immune development, endocrine signaling, and neurologic signaling.

Flavonoids can play an important role in the prevention and amelioration of intestinal barrier permeabilization. The microbiota in the gut can metabolobize flavoinoids releasing absorbable bioactives. These bioactives can restore maintain and/or restore the nutrient balance. Accordingly, flavonoids have the capacity to inhibit inflammation, modulate select signaling cascades, and regulate cellular redox status. However, the large number of existing flavonoids with different chemical/spatial structures, and the multiple metabolites generated at the large intestine by the microbiota, rule out the possibility of simple generalizing the health benefits and mechanisms of action of flavonoids as a class.

Anthocyanins (AC) constitute one of the major flavonoid subgroups, which are present and provide color to many fruits and vegetables (e.g. berries, red cabbage, black rice). Several different AC exist, differing in the number and substitution of hydroxyl groups, the bonded sugar moieties, carboxylates type, and bonding to sugars. A portion of the different classes of anthocyanins includes: cyanidins, delphinidins, peonidins, petunidins, and malvidins.

Current evidence indicates that parental AC are present throughout the length of the gastrointestinal tract. At the colon, AC's can be metabolized by microorganisms in the gut to several metabolites. Thus, in theory, the small intestine and to a lesser extent the large intestine can be exposed to significant amounts of dietary parental AC. Despite the potential for benefits of dietary AC on the gastrointestinal system, the particulars under conditions of increased intestinal permeability, are mostly unknown. AC could exert beneficial effects at the gastrointestinal tract through direct and indirect effects. The indirect effects can be related to the potential capacity of AC to modulate the microbiota, affecting microbiota-mediated AC metabolism. Both a “healthier” microbiota and the formation of select active metabolites could in turn mediate AC indirect effects on intestinal health and lead to systemic effects.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

The present disclosure relates to compositions and methods for improving intestinal health in a subject. In one example, an intestinal health promoting composition is presented. The composition can include a combination of cyanidins and delphinidins (classes of anthocyanins), in an amount sufficient to treat intestinal hyperpermeability. In one example, the cyanidins and the delphinidins can be collectively present in an amount that maintains tight junction integrity in the intestinal epithelial cells. In another example, the cyanidins and the delphinidins can be collectively present in an amount that restores tight junction integrity in the intestinal epithelial cells.

Cyanidins and delphinidins can protect intestinal epithelial cells against TNFα including loss of monolayer permeability in transepithelial electrical resistance (TEER) and increased paracellular transport of FITC-dextran. In contrast, malvidin, peonidin, and petunidin do not seem to provide protective actions to intestinal epithelial cells against TNFα induced TEER permeability and do not seem to increase paracellular transport of FITC-dextran. Without being bound by theory the protective effects of the cyanidins and delphinidins, may be due to the presence of a catechol group on the B-ring of the cyanidins and delphinidins. The anthocyanins that the present inventors tested which did not incorporate the B-ring did not exhibit these protective effects. In one example, this activity can be selective. In another example, the protective function can be dose dependent.

In another example, the present disclosure provides, a method of treating a condition or disorder related to gastrointestinal health in a subject comprising maximizing tight junction integrity in epithelial cells of gastrointestinal tract of the subject. In another example, a method of maximizing tight junction integrity in epithelial cells of the gastrointestinal tract of a subject is presented. In yet another example the present disclosure provides, a method of treating intestinal hyperpermeability. In some examples, these methods can be targeted to (i) maintain and/or create a healthy microbiome in the gastrointestinal system, (ii) maintain and/or create an inflammatory balance within the gastrointestinal system, and/or (iii) maintain and/or form intestinal cell barrier integrity.

In some embodiments, an intestinal health promoting composition can include cyanidins, delphinidins, or a combination thereof, and in some embodiments, these agents can be present in a therapeutically effective amount. In one example, the composition can include a combination of cyanidins and delphinidins in an amount sufficient to treat intestinal hyperpermeability. In one example, the cyanidins and the delphinidins can be collectively present in an amount that maintains intestinal permeability. In another example, the cyanidins and the delphinidins can be collectively present in an amount that reduces intestinal hyperpermeability. In yet another example, the cyanidins and delphinidins can be capable of impacting the microbiome and can be capable of providing anti-inflammatory properties.

The amount of cyanidins and delphinidins can vary in the composition. In one example, the cyanidins and the delphinidins can individually or collectively range from about 5 wt % to about 50 wt % of the composition, or an active fraction of the composition. In another example, the cyanidins and the delphinidins can individually or collectively range from about 12 wt % to about 45 wt % of the composition, or an active fraction of the composition. In a further example, the cyanidins and delphinidins can individually or collectively range from about 12 wt % to about 25 wt % of the composition or an active fraction of the composition.

The cyanidins and delphinidins can be derived from various sources. In one example, a source of at least one of the cyanidins and the delphinidins can be derived from a black rice component, a blueberry component, a black current component, a crowberry component, a bilberry component, a black chokeberry component, or a combination thereof. In another example, the source of the cyanidins and the delphinidins can be derived from the black rice component, the blueberry component, and the black current component. In yet another example, the source of the cyanidins and the delphinidins can be derived from the black rice component and the bilberry component. In a further example, the source of the cyanidins and the delphinidins can be derived from the black current component and the blueberry component. In another example, the cyanidins and delphinidins can be artificially created or synthesized (e.g. “synthetic”).

In one example, the composition can include a black rice component. The black rice component can be derived from black rice kernels, black rice concentrate, black rice extract, black rice powder, or a combination thereof. In one example, the black rice component can be a black rice extract. For example, a liquid rice extract can be obtained by concentrating black rice kernels and passing the concentrated black rice kernels through a resin absorption (chromatography column). In one example, the solvents can be water and ethanol. In one example, the column can be eluted with a 70 wt % ethanol and 30 wt % water solution. In another example, the column can be eluted with 75 wt % ethanol and a 25 wt % water solution. The eluent can then be concentrated into a concentrated liquid extract, dried, and packaged. In another example, the black rice extract can be derived from black rice kernels. In one example, the black rice can be derived from Oryza sativa L.

In one example, the black rice extract can make-up about 2.5 wt % to about 20 wt % of the composition, or an active fraction of the composition. In another example, the black rice component can be present from about 10 wt % to about 15 wt % of the composition, or an active fraction of the composition. In a further example, the black rice extract can be present from about 2.5 wt % to about 5 wt % of the composition, or an active fraction of the composition. In yet another example, the black rice component can be present from about 2.5 wt % to about 7.5 wt % of the composition, or an active fraction of the composition.

In one example, the black rice component can have standardized anthocyanin content. In one example, the black rice component can have a standardized anthocyanin content ranging from about 10 wt % to about 30 wt %. In another example, the black rice component can have a standardized anthocyanin content of about 20 wt %. In a further example, the black rice component can have a standardized anthocyanin content of about 25 wt %. In one example, the standard anthocyanin content can be measured by HPLC. In another example, the standard anthocyanin content can be measured by UV.

In another example, the composition can include the blueberry component. The blueberry component can include a member selected from the group consisting of blueberry fruit, blueberry extract, blueberry concentrate, blueberry juice, blueberry powder, or a combination thereof. In one example, the blueberry component can be a blueberry powder. In another example, the blueberry component can be a blueberry juice. In yet another example, the blueberry component can be a blueberry powder derived from blueberry juice. In a further example, the blueberry component can be blueberry extract. For example, a blueberry fruit extract can be obtained by extracting the blueberry fruit with water. The extraction can be filtered and the filtrate can then be washed with water and parsed with 75% ethanol thru resin adsorption. The extract can then be concentrated under vacuum pressure and spray dried. The concentrated extract was then grinded, sieved, and packaged. In one example, the blueberry component can be a bog blueberry component. In one example, the blueberry component can be derived from Vaccinium uliginosum L.

In one example, the blueberry component can range from about 1 wt % to about 30 wt % of the composition or an active fraction of the composition. In another example, the blueberry component can range from about 1 wt % to about 10 wt % of the composition, or an active fraction of the composition. In yet another example, the blueberry component can range from about 25 wt % to about 30 wt % of the composition, or an active fraction of the composition.

In one example, the blueberry component can have standardized anthocyanin content. In one example, the blueberry component can have a standardized anthocyanin content ranging from about 0.5 wt % to about 30 wt %. In another example, the blueberry component has a standardized anthocyanin content ranging from about 0.5 wt % to about 5 wt %. In yet another example, the blueberry component can have a standardized anthocyanin content ranging from about 20 wt % to about 30 wt %. In a further example, the blueberry component can have a standardized anthocyanin content of about 25 wt %. In yet another example, the blueberry component can have a standardized anthocyanin can be about 17% as measured by UV or about 25% as measured by HPLC.

In one example, the composition can include the black current component. In one example, the black current component can include black current fruit, black current extract, black current concentrate, black current juice, black current powder, or a combination thereof. In another example, the black current component can be a black current powder. In yet another example, the black current component can be a black current juice. In a further example, the black current component can be a black current extract. In one example, the black current extract can be extracted using water, a resin exchange, and ethanol. In one example, the black current can be an ethylene oxide free product. In one example, the black current component can be a derived from Ribes nigrum.

In one example, the black current component can be from about 0.5 wt % to about 15 wt % of the composition, or an active fraction. In another example, the black current component can be from about 1 wt % to about 5 wt % of the composition, or an active fraction of the composition. In yet another example, the black current component can be from about 10 wt % to about 15 wt % of the composition, or an active fraction of the composition.

In one example, the black current component can have standardized anthocyanin content. In one example, the black current component can have a standardized anthocyanin content ranging from about 20 wt % to about 40 wt %. In another example, the black current component can have a standard anthocyanin content of about 30 wt %. In a further example, the black current component can have a standardized anthocyanin content ranging from about 2.5 wt % to about 10 wt %. In one example, the black current component can have a standardized anthocyanin content of about 5 wt %.

In one example, the composition can include a crowberry component. In one example, the crowberry component can be crowberry fruit, a crowberry extract, a crowberry concentrate, a crowberry juice, a crowberry powder, or a combination thereof. In another example, the crowberry component can be crowberry fruit. In yet another example, the crowberry component can be crowberry extract. In one example, the crowberry extract can be extracted using water and ethanol. In one example, the crowberry component can be derived from Empetrum nigrum.

In one example, the crowberry component can range from about 1 wt % to about 30 wt % of the composition, or an active fraction of the composition. In another example, the crowberry component can range from about 5 wt % to 25 wt % of the composition, or an active fraction of the composition. In a further example, the crowberry component can range from about 5 wt % to about 15 wt % of the composition, or an active fraction of the composition.

In one example, the crowberry component can have standardized anthocyanin content. In one example, the crowberry component can have a standard anthocyanin content ranging from about 40 wt % to about 50 wt %. In a further example, the crowberry component can have a standardized anthocyanin content of about 46.7 wt %.

In one example, the composition can include a bilberry component. In one example, the bilberry component can be bilberry fruit, bilberry extract, bilberry concentrate, bilberry juice, bilberry powder, or a combination thereof. In one example, the bilberry component can be a bilberry powder. In another example, the bilberry component can be a bilberry extract. In yet another example, the bilberry extract can be extracted with ethanol and water. In one example, the ethanol to water can have an extract ratio of 150:1. In one example, the bilberry component can be derived from Vaccinium myrtillus.

The amount of the bilberry component in the composition can vary. In one example, the bilberry component can range from about 0.5 wt % to about 30 wt % of the composition, or an active fraction of the composition. In another example, the bilberry component can range from about 2 wt % to about 20 wt % of the composition, or an active fraction of the composition. In a further example, the bilberry component can range from about 5 wt % to about 15 wt % of the composition, or an active fraction of the composition.

In one example, the bilberry component can have standardized anthocyanin content. In one example, the bilberry component can have a standardized anthocyanin content ranging from about 1 wt % to about 40 wt %. In another example, the bilberry component can have a standardized anthocyanin content ranging from about 5 wt % to about 25 wt %. In yet another example, the bilberry component can have a standardized anthocyanin content ranging from about 20 wt % to about 40 wt %. In a further example, the bilberry component can have an anthocyanin content of about 36 wt % as measured by HPLC or about 25 wt % anthocyanins as measured by UV.

In one example, a source of at least one of the cyanidins and the delphinidins can be derived from a black rice component, a blueberry component, and a black current component. In another example, the composition can include a black rice component, a blueberry component, and a black current component at a ratio of about 1:1:1. In another example, the black rice component, the blueberry component, and the black current component can have a ratio of about 1:1.4:4.3. In yet another example, the black rice component, the blueberry component, and the black current component can have a ratio of about 1:2:4.

In one example, the composition can further include a prebiotic ingredient, or blend. Prebiotics can be naturally occurring substances from fruit, vegetables, and grain. Prebiotics can support the microbiome and gastrointestinal system by stimulating growth or activity of at least 1 bacterium in the colon.

In one example, the prebiotic, or prebiotic blend can include inulin, fructooligosacharides, or a combination thereof. In one example, the prebiotic, or prebiotic blend can include inulin. In another example, the prebiotic, or prebiotic blend can include fructooligosacharides. In a further example, the prebiotic, or prebiotic blend can include inulin and fructooligosacharides. In some examples, the prebiotic, or prebiotic blend can promote a healthy microbiome by increasing the fuel available to the beneficial bacteria.

In one example of the composition, the prebiotic, or prebiotic blend can include inulin. In one example, the inulin can be a powder. In one example, the inulin can be a chicory inulin. In another example, the inulin can be a chicory inulin having oligosaccharides and polysaccharides with fructose units linked by β (2-1) linkages. In another example, a source of the inulin can be provided by Orafti® GR. Beneo-Orafti, Bleguim. In some examples, a source of the inulin can be derived from banana, onion, flour, garlic, asparagus, wheat, rye, leeks, chicory root, sugar beets, or a combination thereof. In one example, the inulin can be recovered from the source by diffusion in hot water. In some examples, the inulin can be hydrolyzed or partially hydrolyzed with an enzymatic treatment.

In one example, the inulin can be present at various amounts in the composition. In one example, the inulin can range from about 15 wt % to about 60 wt % of the composition, or an active fraction of the composition. In another example, the inulin can range from about 15 wt % to about 25 wt % of the composition, or an active fraction of the composition. In yet another example, the inulin can range from about 40 wt % to about 60 wt % of the composition, or an active fraction of the composition.

In one example, the composition can include fructooligosaccharides (FOS). In one example, the FOS can be short chain FOS (having a degree of polymerization (DP) of <5). Fructooligosaccharides can be derived from a variety of sources, including grains, fruits, and vegetables. In one example, the short chain FOS can be derived from sucrose. In another example, the short chain FOS can be derived from sugarcane. In another example, the short chain FOS can be derived from a non-GMO source. In yet another example, the FOS can be galactooligosaccharides (GOS).

In one example, the FOS can be present in varying amounts in the composition. In one example, the FOS can range from about 10 wt % to about 40 wt % of the composition, or an active fraction of the composition. In another example, the FOS can range from about 10 wt % to about 20 wt % of the composition, or an active fraction of the composition. In yet another example, the fructooligosaccharides can range from about 25 wt % to about 40 wt % of the composition, or an active fraction of the composition.

In one example, the composition can include inulin and fructooligosacharides. When present, in one example, the inulin and fructooligosacharides can collectively range from about 55 wt % to about 95 wt % of the composition, or an active fraction of the composition. In another example, the inulin and fructooligosaccharides can collectively range from about 70 wt % to about 90 wt % of the composition, or an active fraction of the composition.

The ingredients above can be combined in a composition in any variety of manners. Some, exemplary compositions are tabled below. In the tables below, some of these examples include excipients such as, silicon dioxide, while others display only the active faction of the composition.

TABLE 1
Exemplary Formulation

| Ingredient | Amount (mg) | Total wt % |
|---|---|---|
| Black Rice Extract (25% AC) | 770 | 12.28% |
| Bilberry Extract (36% AC) | 122.22 | 1.95% |
| Galactooligosacharides | 2,769.23 | 44.16% |
| Short chain Fructooligosacharides | 1,100 | 17.54% |
| Inulin | 1,500 | 23.92% |
| Silicon Dioxide | 10 | 0.16% |
| Total | 6271.45 | 100.00% |

TABLE 2
Exemplary Formulation

| Ingredient | Amount (mg) | Total wt % |
|---|---|---|
| Black Current Extract (5% AC) | 1320 | 13.89% |
| Blueberry Juice Powder (0.8% AC) | 2750 | 28.95% |
| Galactooligosacharides | 2,769.23 | 29.15% |
| Short chain Fructooligosacharides | 1,100 | 11.58% |
| Inulin | 1,500 | 15.79% |
| Silicon Dioxide | 60.769 | 0.64% |
| Total | 9499.999 | 100.00% |

TABLE 3
Exemplary Formulation

| Ingredient | Amount (mg) | Total wt % |
|---|---|---|
| Blueberry Extract (24% AC) | 144 | 3.63% |
| Black Current Extract (29% AC) | 206 | 5.19% |
| Black Rice Extract (19% AC) | 618 | 15.57% |
| Short chain Fructooligosacharides | 1,100 | 27.72% |
| Inulin | 1,900 | 47.88% |
| Total | 3,968 | 100.00% |

TABLE 4
Exemplary Formulation

| Ingredient | Amount (mg) | Total wt % |
|---|---|---|
| Black Rice Extract | 120 | 4.12% |
| Black Current Extract | 60 | 2.06% |
| Blueberry Extract | 35 | 1.20% |
| Fructooligosacharides | 1,100 | 37.74% |
| Inulin | 1,600 | 54.89% |
| Total | 2,915 | 100.00% |

In some examples, the composition can be further formulated to include additional excipients.

In one example, the composition can further include epicatechins, catechins, or a combination thereof. In some formulations, epicatechin and catechins can act as NADPH oxidase inhibitors.

In one example, the composition can include a pharmaceutically acceptable carrier. In another example, the composition can include a sweetener, a preservative, a flavoring, a thickener, or a combination thereof. In yet another example, the composition can further include coatings, isotonic agents, absorption delaying agents, binders, adhesives, lubricants, disintergrants, coloring agents, flavoring agents, sweetening agents, absorbents, detergents, emulsifying agents, antioxidants, vitamins, minerals, proteins, fats, carbohydrates, or a combination thereof. In some examples, the formulation can include polymers for sustained release of a given compound. Nearly any number or type of ingredients required in order to produce a specifically desired composition or formulation can be used.

In one example, the composition can be in the form of an oral dosage formulation. In one example, the oral dosage form comprises a capsule, a tablet, a powder, a beverage, a syrup, a gummy, a wafer, a confectionary, a suspension, or a food. In another example, the oral dosage form can be in the form of a capsule, a tablet, a soft gel, a lozenge, a sachet, a powder, a beverage, a syrup, a suspension, or a food. In another example, the oral dosage formulations can be formulated into a food or drink, and provided, for example, as a snack bar, a cereal, a drink, a gum, or in any other easily ingested form. In one example, the oral dosage form can be incorporated into a liquid beverage such as water, milk, juice, or soda. In another example, the oral dosage form can be formulated into a nutritional beverage. The nutritional beverage can be in a premixed formulation or can be a powdered mix in that can be added to a beverage. In another example, the powder mix in can be in the form of granules. In another example, the composition can be a powder that can be sprinkled onto food.

In one example, the oral dosage form can be designed to be administered to a subject in need thereof once per day. In one example, the oral dosage form can be designed to be administered to the subject in the morning. In another example, the oral dosage form can be administered in the afternoon, or in the evening. In yet another example, the dosage form can be designed to be administered on an on and off and on again basis. For example, the dosage form can be administered for 2 days on and 1 day off, 3 days on with 2 days off, 3 days on with 4 days off, 4 days on with 3 days off, 5 days on with 2 days off; or 6 days on with 1 day off, with each of these regimens repeating consecutively for a length of time. In another example, the dosage regimen can alternate on and off every day. The period of dosage can also vary. For example, the dosage form can be designed to be administered for a two-weeks, a three-weeks, a months, 6 weeks, two months, three months, four months, 5 months, 6 months, a year, a year and a half, or indefinitely.

The oral dosage form can include any of the compositions identified herein. In one example, the oral dosage form comprises a member selected from the group consisting of a black rice component, a blueberry component, a black current component, a crowberry component, a bilberry component, black chokeberry component, or a combination thereof.

In one example, the oral dosage form can include the black rice component and the black rice component can range from about 500 mg to about 800 mg of the oral dosage form. In another example, the black rice component can have a standardized anthocyanin content of about 15 wt % to about 30 wt %.

In one example, the oral dosage form can include the blueberry component and the blueberry component can range from about 100 mg to about 3,000 mg of the oral dosage form. In another example, the blueberry component can range from about 50 mg to about 500 mg. In a further example, the blueberry component can range from about 2,000 mg to about 3,000 mg. In yet another example, the blueberry component can have a standardized anthocyanin content of about 0.5 wt % to about 25 wt %.

In one example, the oral dosage form can include the black current component and the black current component can range from about 200 mg to about 3,000 mg of the oral dosage form. In another example, the black current component can range from about 50 mg to about 500 mg. In a further example, the black current component can range from about 2,000 mg to about 3,000 mg. In yet another example, the black current component has a standardized anthocyanin content of about 2.5 wt % to about 30 wt %.

In one example, the oral dosage form can include the crowberry component and the crowberry component can range from about 100 mg to about 1,000 mg of the oral dosage form. In one example, the crowberry component has a standardized anthocyanin content from about 1 wt % to about 50 wt %. In another example, the crowberry component has a standardized anthocyanin content from about 1 wt % to about 30 wt %. In yet another example, the crowberry component has a standardized anthocyanin content ranging from about 40 wt % to about 50 wt %.

In another example, the oral dosage form can include the bilberry component and the bilberry component can range from about 100 mg to about 700 mg of the oral dosage form. In one example, the bilberry component can have a standardized anthocyanin content of about 30 wt % to about 40 wt %.

In a further example, the oral dosage form can include the black chokeberry component and the black chokeberry component can range from about 50 mg to about 700 mg. In another example, the black chokeberry component can range from about 100 mg to about 600 mg. In yet another example, the black chokeberry component can range from about 200 mg to about 500 mg. In one example, the black chokeberry component can have a standardized anthocyanin content of about 1 wt % to about 35 wt %.

In one example, the oral dosage form can further include the prebiotic, or prebiotic blend. The prebiotic, or prebiotic blend can include inulin, fructooligosaccharides, or a combination thereof. In one example, the inulin can be present in the oral dosage in an amount of from about 1 grams to 2 grams. In another example, the inulin can provide from about 1 gram to about 2 grams of fiber in the oral dosage form.

In one example, the oral dosage form can include a fructooligosaccharides (FOS). In one example, the FOS can range from about 1 gram to about 1. 5 grams of the oral dosage form. In another example, the FOS can range from about 3 grams to about 4 grams of the oral dosage form.

The oral dosage from can provide various amounts of anthocyanins. In one example, the oral dosage from can collectively provide from about 200 mg to about 300 mg of anthocyanins. In another example, the oral dosage form can provide from 50 mg to 100 mg of anthocyanins. In one example, the oral dosage from can provide about 80 mg of anthocyanins. In another example, the oral dosage from can provide about 215 mg of anthocyanins.

The oral dosage form can also provide various amounts of fiber. In one example, the oral dosage form can collectively provide from about 1.5 grams to about 3 grams of fiber. In one example, the oral form can provide about 2.6 g, 2.7 g, or 2.9 g, of fiber.

Methods of making the present compositions are also encompassed by the present invention. In one embodiment, the method can include combining the ingredients, whether raw or extracted, and processing the combined ingredients into the desired form of the composition. In one example, the desired form of the composition can be a tablet, capsule, powder, food, or beverage. Individuals skilled in the art are of the procedures that can be used to combine the components.

Further presented herein, are methods of treating intestinal hyperpermeability in a subject. In one example, the method can include administering an intestinal health promoting composition to the subject. In one example, the intestinal health promoting composition can be as described herein.

In another example, the intestinal health promoting composition can be administered to the subject on a daily basis. In one example, the administration can occur in the morning. In yet another example, the administration can occur for an extended period of time, such as, every day for at least 3 weeks, at least 4 weeks, at least 6 weeks, at least 8 weeks, at least 12 weeks, at least 6 months, at least 9 months, at least a year, at least two years, any period in-between these, or indefinitely.

Additionally presented, are methods of treating a condition or disorder related to gastrointestinal health in a subject. In one example, the method can include maximizing tight junction integrity in epithelial cells of gastrointestinal tract of the subject. In another example, the method can include reducing intestinal hyperpermeability in the subject.

In one example, the method can improve the gastrointestinal health of the subject. The improvement in gastrointestinal health can vary.

In one example, the improvement can include an improvement in the subject's bowel habits when compared to the subject's bowel habits before administering the method. In another example, the improvement in the subject's bowel habits can include a decrease in straining during and after bowel movements. In yet another example, the improvement can be a reduction in bloating, discomfort, gas, or a combination thereof. In a further example, the improvement can be reducing the gut permeability of the subject. In yet another example, the improvement can be a reduction in the symptoms/occurrence of leaky gut syndrome.

In a further example, the improvement in gastrointestinal health can be a reduction in intestinal dysbosis. In one example, the improvement can be evidenced by reduced calprotectin fecal levels. In yet another example, the improvement can be an increase short chain fatty acid levels.

In one example of the method, the condition or disorder can include inflammation, inflammatory bowel disease, irritable bowel syndrome, chronic intestinal diseases, celiac disease, Crohn's disease, ulcerative colitis, food intolerances, dyspepsia, low levels of chronic intestinal inflammation, gastrointestinal infections, or a combination thereof.

In another example, the condition or disorder can be inflammation and the inflammation can be reduced in the subject's gastrointestinal tract when compared to the subject's gastrointestinal inflammation before administering the method. In another example, the reduction can be about a 50% reduction in inflammation. In yet another example, the reduction can be about a 60% reduction in inflammation. In a further example, the reduction can be about a 70% reduction in inflammation. In one example, the reduction can be up to a 73% reduction in inflammation. In a further example, supplementation for a 3 week period can result in a modest reduction of inflammatory biomarkers.

In a further example, the condition or disorder can include insufficient absorption of nutrients, endotoxemia, intestinal hyperpermeability, or a combination thereof.

In another example, the condition or disorder can include obesity, obesity associated pathologies, allergies, cardiovascular conditions, type I diabetes, type II diabetes, rheumatoid arthritis, insulin resistance, cancer, metabolic syndrome, asthma, neurodegenerative diseases, or a combination thereof.

In one example, the condition or disorder can be a cardiovascular condition or disorder. In one example, the cardiovascular condition or disorder can be an increase in high-density lipoprotein cholesterol for subjects that are not taking cardiovascular medications. In another example, the cardiovascular condition or disorder can be a decrease in HbA1c levels. In yet another example, the decrease in HbA1c levels can be from pre-diabetic levels to normal levels as measured by reducing HbA1c levels from a range of 6-6.4% to below 6%. In a further example, the decrease in HbA1c levels can be from diabetic levels to pre-diabetic levels. This is a reduction from levels above 6.5% to levels between 6% and 6.4%.

In a further example, the cardiovascular condition or disorder can be related to an increase in plasma zonulin levels. In one example, administering the method can decrease plasma zonulin levels when compared to plasma zonulin levels prior to administering the method. An increase in plasma zonulin can be indicative of intestinal permeability. Zonulin can modulate intestinal permeability by disassembling tight junctions and allowing larger molecules, such as, lactulose to pass through. Administering the compositions herein, can maximize tight junction integrity; thereby, minimizing the amount of zonulin that passes through the intestinal lining.

In another example, the condition can be peripheral insulin resistance and the method can reduce peripheral insulin resistance. In another example, the condition or disorder can be type I diabetes or type II diabetes.

In yet another example, the condition or disorder can be a nitric oxide related disorder, expression of iNOS, expression of COX-2, NADPH oxidase, or a combination thereof. In one example, the condition or disorder can stem from pathogens, antigens, and pro-inflammatory factors that can pass through the tight junctions.

In one example, the method can include maximizing tight junction integrity or reducing intestinal hyperpermeability. Maximizing tight junction integrity can include protecting the gastrointestinal tract of the subject from TNFα induced permealization of a monolayer of the epithelial cells. In another example, the amount of the protecting can be concentration dependent on an amount of cyanidins and delphinidins in the subject's gastrointestinal tract. In one example, the amount of protection is dose dependent.

In yet another example, the method can further include increasing transepithelial electrical resistance in the epithelial cells. In a further example, the method can include increasing FITC dextran paracellular transport.

In one example, the condition or disorder can stem from pro-inflammatory factors. In one example, the pro-inflammatory factors can be advanced glycation end products. In another example, the pro-inflammatory factors can be lipopolysaccharides. In a further example the pro-inflammatory factors can include cytokines tumor necrosis alpha (TNF-α), IL-6, or a combination thereof.

In one example of the method, the condition or disorder can relate to conditions or disorders associated with signaling pathways NF-kB, ERK1/2, or a combination thereof.

In one example, maximizing of the tight junction integrity can mitigate high fat induced intestinal permeabilization.

In another example, the epithelial cells can include Caco-2 cell monolayers.

In another example, the method can include optimizing a balance of gut microbiota in the gastrointestinal tract. In one example, optimizing the balance of gut microbiota can include increasing commensal bacteria levels in the gastrointestinal tract when compared to levels of commensal bacteria in the gastrointestinal tract prior to administering the method. In another example, the commensal bacteria can belong to bifidobacteria genus. In one example, the commensal bacteria can belong to bacteroidetes phylum. In another example, the commensal bacteria can be bacterdies caccae, bacteriodes umformis, or a combination thereof. In yet another example, the increase in the commensal bacteria after 8 weeks of daily administering the method to the subject can be at least 20%. In another example, the increase in commensal bacteria after 8 weeks of daily administering the method to the subject can be about 5%, can about 10%, can be about 15%, or can be about 25%.

In one example of the method, optimizing the balance of gut microbiota can include increasing diversity of bacteria when compared to the diversity of bacteria present in the gut prior to administering the method. In one example, the diversity of bacteria can include at least 200 strains. In another example, the method can include decreasing harmful gut bacteria when compared to a level of harmful gut bacteria present prior to administering the method. In one example, the harmful gut bacteria can include firmicutes. In some studies firmicutes have been found to make up a higher portion of the gut microbiome in obese individuals. In yet another example, the decreasing of the firmicutes after 8 weeks of daily administering the method to the subject can be greater than a 15% reduction in firmicutes levels. In a further example, the decreasing of the firmicutes after 8 weeks of daily administering the method to the subject can be greater than a 5% reduction, about a 10% reduction, about a 12% reduction, about a 15% reduction, or about a 20% reduction.

In one example, the method can include optimizing the gut microbiota by altering the firmicutes:bacteriodetes ratio. In one example, the ratio of firmicutes:bacteriodetes can be decreased by approximately 3% after 8 weeks of administering the method to the subject. The firmicutes:bacteriodetes ratio can be a contributing factor in obesity. Individuals with a high body mass index display gut microbiota differences at the phylum level and can have high firmicutes concentrations and can have low levels of bacteriodetes. It is noted, that changes in the microbiota have not been correlated to caloric content but rather to body mass index. Accordingly, altering this ratio can allow for weight loss in the individual if administered for a period of time.

In another example of the method for decreasing harmful gut bacteria, the harmful gut bacteria can include Actinobacteria. In one example, the reduction of the Actinobacteria after 8 weeks of daily administering the method to the subject can be at least about 5%.

In yet another example of the method for decreasing harmful gut bacteria, the harmful gut bacteria can include Helicobacter pylori. Helicobacter pylori can be associated with ulcers and heartburn. In a further example of the method, the harmful gut bacteria can include Clostridium. In yet another example, the harmful gut bacteria can include Klebisella.

In one example, the method can further include providing a fuel source for commensal bacteria in the gut microbiome. In doing this, the method can generate greater portions of commensal bacteria which can ultimately lead to systemic health benefits.

In another example, a method of maximizing tight junction integrity in epithelial cells of gastrointestinal tract of the subject is provided. In one example, of this method the improvement in tight junction integrity can include restoring tight junction integrity. In another example of this method, the improvement can include maintaining tight junction integrity. In a further example, this method can provide systemic health benefits. These health benefits can include improvements in conditions or disorders, such as, celiac disease, IBS, Crohn's disease, ulcerative colitis, food intolerances, allergies, dyspepsia, low levels of chronic inflammation, obesity, type I diabetes, type II diabetes, rheumatoid arthritis, insulin resistance, metabolic syndrome, asthma, atopy, leaky intestinal barrier, tight junction barrier dysfunction, plasma glucose levels, plasma free fatty acid levels, reduced high density lipoprotein levels, hepatic steatosis, firmicutes:bacteriodetes levels in the gut, abdominal bloating, abdominal gas, abdominal pain, bowel function, growth of favorable intestinal bacteria, short chain fatty acid production, plasma zonulin levels, HbA1c levels, diabetes, prediabetes, nutrient absorption, and combinations thereof.

The benefits of the various methods described above can be achieved by administering a gastrointestinal health promoting composition described above, to a subject. In one example, the compositions can be administered on a daily basis for an extended period of time. In another example, the compositions can be administered on an on and off and on again basis based on a dosing regimen. For example, the dosage regimen can be 2 days of administration followed by 1 day without administration. In another example, the dosage regimen could be 5 days of administration followed by two days without administration. In yet another example, the dosage regimen could be 3 days of administration followed by 1, 2, 3, or 4 days without administration. In a further example, the dosage regimen could be 4 days of administration followed by 1, 2, 3, or 4 days without administration. In some examples, the extended period of time can vary. In one example, the extended period of time can be about 4 weeks. In another example, the extended period of time can be about 6 weeks, about 8 weeks, about 12 weeks, 16 weeks, 20 weeks, about 6 months, about 9 moths, about 1 year, or a period of time greater than a year. In some examples, the benefits of administering the method for a period of time can increase with longer periods of administration.

### Embodiments

In one embodiment presented herein, is an intestinal health promoting composition, comprising a combination of cyanidins and delphinidins, in an amount sufficient to treat intestinal hyperpermeability.

In one embodiment of the composition, the cyanidins and the delphinidins are collectively present in an amount that maintains intestinal permeability.

In one embodiment of the composition, the cyanidins and the delphinidins are collectively present in an amount that reduces intestinal hyperpermeability.

In one embodiment of the composition, can include a source of at least one of the cyanidins and the delphinidins that are derived from a black rice component, a blueberry component, a black current component, a crowberry component, a bilberry component, black chokeberry component, or a combination thereof.

In one embodiment of the composition, the source of the cyanidins and the delphinidins are derived from the black rice component, the blueberry component, and the black current component.

In one embodiment of the composition, the composition comprises the black rice component and the black rice component is derived from a member selected from the group consisting of black rice kernels, black rice concentrate, black rice extract, black rice powder, or a combination thereof.

In one embodiment of the composition, the black rice component is a black rice extract.

In one embodiment of the composition, the black rice extract is derived from black rice kernels.

In one embodiment of the composition, the black rice component comprises from about 2.5 wt % to about 20 wt % of the active fraction of the composition.

In one embodiment of the composition, the black rice component comprises from about 10 wt % to about 15 wt % of the active fraction of the composition.

In one embodiment of the composition, the black rice component comprises from about 2.5 wt % to about 7.5 wt % of the active fraction of the composition.

In one embodiment of the composition, the black rice component has a standardized anthocyanin content ranging from about 10 wt % to about 30 wt %.

In one embodiment of the composition, the black rice component has a standardized anthocyanin content of about 20 wt %.

In one embodiment of the composition, the black rice component has a standardized anthocyanin content of about 25 wt %.

In one embodiment of the composition, the black rice component is derived from Oryza sativa L.

In one embodiment of the composition, the composition comprise the blueberry component and the blueberry component comprises a member selected from the group consisting of blueberry fruit, blueberry extract, blueberry concentrate, blueberry juice, blueberry powder, or a combination thereof.

In one embodiment of the composition, the blueberry component is a blueberry powder.

In one embodiment of the composition, the blueberry component is a blueberry juice.

In one embodiment of the composition, the blueberry component comprises from about 1 wt % to about 30 wt % of the active fraction of the composition.

In one embodiment of the composition, the blueberry component comprises from about 1 wt % to about 10 wt % of the active fraction of the composition.

In one embodiment of the composition, the blueberry component comprises from about 25 wt % to about 30 wt % of the active fraction of the composition.

In one embodiment of the composition, the blueberry component has a standardized anthocyanin content ranging from about 0.5 wt % to about 30 wt %.

In one embodiment of the composition, the blueberry component has a standardized anthocyanin content ranging from about 0.5 wt % to about 5 wt %.

In one embodiment of the composition, the blueberry component has a standardized anthocyanin content ranging from about 20 wt % to about 30 wt %.

In one embodiment of the composition, the blueberry component is derived from Vaccinium uliginosum L.

In one embodiment of the composition, the composition comprises the black current component and the black current component comprises a member selected from the group consisting of black current fruit, black current extract, black current concentrate, black current juice, black current powder, or a combination thereof.

In one embodiment of the composition, the black current component is a black current extract.

In one embodiment of the composition, the black current component comprises from about 0.5 wt % to about 15 wt % of the active fraction.

In one embodiment of the composition, the black current component comprises from about 1 wt % to about 5 wt % of the active fraction.

In one embodiment of the composition, the black current component has a standardized anthocyanin content ranging from about 20 wt % to about 40 wt %.

In one embodiment of the composition, the black current component has a standard anthocyanin content of about 30 wt %.

In one embodiment of the composition, the black current component is derived from Ribes nigrum.

In one embodiment of the composition, the composition comprises the crowberry component and the crowberry component comprises a member selected from the group consisting of crowberry fruit, crowberry extract, crowberry concentrate, crowberry juice, crowberry powder, or a combination thereof.

In one embodiment of the composition, the crowberry component comprises crowberry fruit.

In one embodiment of the composition, the crowberry component comprises crowberry extract.

In one embodiment of the composition, the crowberry component comprises from about 1 wt % to about 30 wt % of the active fraction of the composition.

In one embodiment of the composition, the crowberry component comprises from about 5 wt % to about 25 wt % of the composition.

In one embodiment of the composition, the crowberry component has a standard anthocyanin content ranging from about 40 wt % to about 50 wt %.

In one embodiment of the composition, the crowberry component has a standardized anthocyanin content of about 46.7 wt %.

In one embodiment of the composition, the crowberry component is derived from Empetrum nigrum.

In one embodiment of the composition, the composition comprises the bilberry component and the bilberry component comprises a member selected from the group consisting of bilberry fruit, bilberry extract, bilberry concentrate, bilberry juice, bilberry powder, or a combination thereof.

In one embodiment of the composition, the bilberry component comprises bilberry extract.

In one embodiment of the composition, the bilberry component ranges from about 0.5 wt % to about 30 wt % of the active fraction of the composition.

In one embodiment of the composition, the bilberry component ranges from about 2 wt % to about 20 wt % of the composition.

In one embodiment of the composition, the bilberry component has a standardized anthocyanin content ranging from about 1 wt % to about 30 wt %.

In one embodiment of the composition, the bilberry component has a standardized anthocyanin content ranging from about 5 wt % to about 15 wt %.

In one embodiment of the composition, the bilberry component comprises 36 wt % anthocyanins as measured by HPLC or 25 wt % anthocyanins as measured by UV.

In one embodiment of the composition, the bilberry component is derived from Vaccinium myrtillus.

In one embodiment of the composition, the source of at least one of the cyanidins and the delphinidins is derived from a black rice component, a blueberry component, and a black current component.

In one embodiment of the composition, the black rice component, the blueberry component, and the black current component have a ratio of about 1:1:1.

In one embodiment of the composition, the black rice component, the blueberry component, and the black current component have a ratio of about 1:1.4:4.3.

In one embodiment of the composition, the composition further comprises a prebiotic blend.

In one embodiment of the composition, the prebiotic blend comprises inulin.

In one embodiment of the composition, the inulin is a chicory inulin having oligosaccharides and polysaccharides with fructose units linked by p (2-1) linkages.

In one embodiment of the composition, the inulin is derived from banana, onion, flour, garlic, asparagus, wheat, rye, leeks, chicory root, sugar beets, or a combination thereof.

In one embodiment of the composition, the inulin comprises from about 15 wt % to about 60 wt % of the composition.

In one embodiment of the composition, the inulin comprises from about 15 wt % to about 25 wt % of the composition.

In one embodiment of the composition, the inulin comprises from about 40 wt % to about 60 wt % of the composition.

In one embodiment of the composition, the prebiotic blend comprises fructooligosaccharides.

In one embodiment of the composition, the fructooligosaccharides are short chain fructooligosaccharides (DP≤5).

In one embodiment of the composition, the short chain fructooligosaccharides are derived from sucrose.

In one embodiment of the composition, the short chain fructooligosaccharides are derived from sugarcane.

In one embodiment of the composition, the fructooligosaccharides comprise from about 10 wt % to about 40 wt % of the active fraction of the composition.

In one embodiment of the composition, the fructooligosaccharides comprise from about 10 wt % to about 20 wt % of the active fraction of the composition.

In one embodiment of the composition, the fructooligosaccharides comprise from about 25 wt % to about 40 wt % of the active fraction of the composition.

In one embodiment of the composition, the fructooligosaccharides comprise galactooligosaccharides.

In one embodiment of the composition, the composition further comprises a prebiotic blend of inulin and fructooligosacharides.

In one embodiment of the composition, the inulin and fructooligosacharides collectively comprise from about 55 wt % to about 95 wt % of the composition.

In one embodiment of the composition, a combine source of the cyanidins and the delphinidins comprise from about 5 wt % to about 50 wt % of the composition.

In one embodiment of the composition, the composition further comprises a pharmaceutically acceptable carrier.

In one embodiment of the composition, the composition further comprises a sweetener, a preservative, a flavoring, a thickener, or a combination thereof.

In one embodiment of the composition, the composition is an oral dosage form.

In one embodiment of the composition, the oral dosage form comprises a capsule, a tablet, a powder, a beverage, a syrup, a gummy, a wafer, a confectionary, a suspension, or a food.

In one embodiment of the composition, the oral dosage form comprises a powder.

In one embodiment of the composition, the oral dosage form is designed to be administered to a subject in need thereof once per day.

In one embodiment of the composition, the oral dosage form is designed to be administered to the subject at morning.

In one embodiment of the composition, the oral dosage form comprises a member selected from the group consisting of a black rice component, a blueberry component, a black current component, a crowberry component, a bilberry component, black chokeberry component, or a combination thereof.

In one embodiment of the composition, the oral dosage form comprises the black rice component and the black rice component ranges from about 500 mg to about 800 mg of the oral dosage form.

In one embodiment of the composition, the black rice component has a standardized anthocyanin content of about 15 wt % to about 30 wt %.

In one embodiment of the composition, the oral dosage form comprises the blueberry component and the blueberry component comprises from about 100 mg to about 3,000 mg of the oral dosage form.

In one embodiment of the composition, the blueberry component has a standardized anthocyanin content of about 0.5 wt % to about 25 wt %.

In one embodiment of the composition, the oral dosage form comprises the black current component and the black current component comprises from about 200 mg to about 3,000 mg of the oral dosage form.

In one embodiment of the composition, the black current component has a standardized anthocyanin content of about 2.5 wt % to about 30 wt %.

In one embodiment of the composition, the oral dosage form comprises the crowberry component and the crowberry component comprises from about 100 mg to about 1,000 mg of the oral dosage form.

In one embodiment of the composition, the crowberry component has a standardized anthocyanin content of about 1 wt % to about 30 wt %.

In one embodiment of the composition, the oral dosage form comprises the bilberry component and the bilberry component ranges from about 100 mg to about 700 mg of the oral dosage form.

In one embodiment of the composition, the bilberry component has a standardized anthocyanin content of about 30 wt % to about 40 wt %.

In one embodiment of the composition, the oral dosage form comprises the black chokeberry component and the black chokeberry component ranges from about 50 mg to about 700 mg of the oral composition.

In one embodiment of the composition, the black chokeberry component has a standardized anthocyanin content of about 1 wt % to about 35 wt %.

In one embodiment of the composition, the oral dosage form further comprises a prebiotic blend.

In one embodiment of the composition, the prebiotic blend comprise from about 1 grams to 2 grams of the oral dosage form.

In one embodiment of the composition, the prebiotic blend provides from about 1 gram to about 2 grams of fiber in the oral dosage form.

In one embodiment of the composition, the oral dosage form further comprises a fructooligosaccharide.

In one embodiment of the composition, the fructooligosaccharide comprises from about 1 gram to about 1. 5 grams of the oral dosage form.

In one embodiment of the composition, the fructooligosaccharide comprises from about 3 grams to about 4 grams of the oral dosage form.

In one embodiment of the composition, the oral dosage form comprises from about 200 mg to about 300 mg of anthocyanins.

Also presented herein in one embodiment, is a method of treating intestinal hyperpermeability.

In one embodiment of the method, the method can include administering an intestinal health promoting composition to a subject.

In one embodiment of the method, the intestinal health promoting composition comprises a combination of cyanidins and delphinidins, in an amount sufficient to treat intestinal hyperpermeability.

In one embodiment of the method, the intestinal health promoting composition further comprises a prebiotic blend.

In one embodiment of the method, the intestinal health promoting composition further comprises a fructooligosaccharide.

In another embodiment of the method, the administering of the intestinal health promoting composition can be on a daily basis.

In one embodiment of the method, the administering can occur in morning.

In one embodiment of the method, the administering can occur for at least 3 weeks.

Further presented herein, in one embodiment, is a method of treating a condition or disorder related to gastrointestinal health in a subject comprising maximizing tight junction integrity in epithelial cells of gastrointestinal tract of the subject.

In one embodiment of the method, the gastrointestinal health of the subject is improved when compared to the health of the gastrointestinal system in the subject prior to administering the method.

In one embodiment of the method, the improved gastrointestinal health of the subject comprises an improvement in the subject's bowel habits when compared to the subject's bowel habits prior to administering the method.

In one embodiment of the method, the improved gastrointestinal health of the subject comprises reducing an occurrence of bloating, discomfort, gas, or a combination thereof in the subject when compared to the occurrence of bloating, discomfort, gas, or the combination thereof prior to administering the method.

In one embodiment of the method, the improved gastrointestinal health of the subject comprises reducing intestinal hyperpermeability.

In one embodiment of the method, the improved gastrointestinal health of the subject comprises an improvement in intestinal dysbiosis when compared to a level of intestinal dysbiosis prior to administering the method.

In one embodiment of the method, the improved gastrointestinal health of the subject comprises reduced calprotectin fecal levels.

In one embodiment of the method, the improved gastrointestinal health of the subject comprises an increase short chain fatty acid levels.

In one embodiment of the method, the condition or disorder comprises inflammation, inflammatory bowel disease, irritable bowel syndrome, chronic intestinal diseases, celiac disease, Crohn's disease, ulcerative colitis, food intolerances, dyspepsia, low levels of chronic intestinal inflammation, gastrointestinal infections, or a combination thereof.

In one embodiment of the method, the inflammation is reduced overall.

In one embodiment of the method, the inflammation is reduced by up to 73%.

In one embodiment of the method, supplementation for 3 weeks with a gastrointestinal health promoting composition results in a modest reduction in inflammatory biomarkers.

In one embodiment of the method, the condition or disorder comprises insufficient absorption of nutrients, endotoxemia, intestinal hyperpermeability, or a combination thereof.

In one embodiment of the method, the condition or disorder comprises obesity, obesity associated pathologies, allergies, cardiovascular conditions, type I diabetes, type II diabetes, rheumatoid arthritis, insulin resistance, cancer, metabolic syndrome, asthma, neurodegenerative diseases, or a combination thereof.

In one embodiment of the method, the condition or disorder is a cardiovascular condition.

In one embodiment of the method, the cardiovascular condition is an increase in high-density lipoprotein cholesterol for subjects that are not taking cardiovascular medications.

In one embodiment of the method, the cardiovascular condition comprises a decrease in HbAlc levels.

In one embodiment of the method, the decrease in HbAlc levels is from pre-diabetic levels to normal levels.

In one embodiment of the method, the cardiovascular condition comprises a decrease in plasma zonulin levels.

In one embodiment of the method, the condition is peripheral insulin resistance and the method reduces peripheral insulin resistance.

In one embodiment of the method, condition or disorder is type I diabetes or type II diabetes.

In one embodiment of the method, the condition or disorder comprises a nitric oxide related disorder, expression of iNOS, expression of COX-2, NADPH oxidase, or a combination thereof.

In one embodiment of the method, the condition or disorder stems from pathogens, antigens, and pro-inflammatory factors that pass through the tight junctions in the epithelial cells of the gastrointestinal tract.

In one embodiment of the method, maximizing tight junction integrity comprises protecting the gastrointestinal tract of the subject from TNFα induced permeabilization of a monolayer of the epithelial cells.

In one embodiment of the method, an amount of the protecting is concentration dependent on an amount of cyanidins and delphinidins in the subject's gastrointestinal tract.

In one embodiment of the method, the method further comprises increasing transepithelial electrical resistance in the epithelial cells.

In one embodiment of the method, the method further comprises increasing FITC dextran paracellular transport.

In one embodiment of the method, the condition stems from pro-inflammatory factors and the pro-inflammatory factors comprise advanced glycation end products.

In one embodiment of the method, the condition stems from pro-inflammatory factors and the pro-inflammatory factors comprise lipopolysaccharides.

In one embodiment of the method, the condition stems from pro-inflammatory factors and the pro-inflammatory factors comprise cytokines tumor necrosis alpha (TNF-α), IL-6, or a combination thereof.

In one embodiment of the method, the condition or disorder relates to conditions or disorders associated with signaling pathways NF-kB, ERK1/2, or a combination thereof.

In one embodiment of the method, the maximizing of the tight junction integrity mitigates high fat induced intestinal permeabilization.

In one embodiment of the method, the epithelial cells comprise Caco-2 cell monolayers.

In one embodiment of the method, the method further comprises optimizing a balance of gut microbiota in the gastrointestinal tract.

In one embodiment of the method, optimizing the balance of gut microbiota comprises increasing commensal bacteria levels in the gastrointestinal tract above a level of the communsual bacteria in the gastrointestinal tract prior to administering the method.

In one embodiment of the method, the commensal bacteria belong to bifidobacteria genus.

In one embodiment of the method, the commensal bacteria belong to bacteroidetes phylum.

In one embodiment of the method, the commensal bacteria comprise bacterdies caccae, bacteriodes uniformis, or a combination thereof.

In one embodiment of the method, the increase in the commensal bacteria after 8 weeks of daily administering the method to the subject in need thereof was at least 20%.

In one embodiment of the method, optimizing the balance of gut microbiota comprises increasing diversity of bacteria.

In one embodiment of the method, the diversity of bacteria comprises at least 200 strains.

In one embodiment of the method, the method further comprises decreasing a level harmful gut bacteria when compared to the level of harmful gut bacteria prior to administering the method.

In one embodiment of the method, the harmful gut bacteria comprise firmicutes.

In one embodiment of the method, the decreasing of the firmicutes after 8 weeks of daily administering the method to the subject was greater than a 15% reduction.

In one embodiment of the method, a ratio of firmicutes:bacteriodetes decreased approximately 3% after 8 weeks of administering the method to the subject.

In one embodiment of the method, the harmful gut bacteria comprise Actinobacteria.

In one embodiment of the method, the decreasing of the Actinobacteria after 8 weeks of daily administering the method to the subject was at least 5%.

In one embodiment of the method, the harmful gut bacteria comprise Helicobacter pylori.

In one embodiment of the method, the harmful gut bacteria comprise Clostridium.

In one embodiment of the method, the harmful gut bacteria comprise Klebisella.

In one embodiment of the method, the method comprises providing a fuel source for commensal bacteria.

### EXAMPLES

### Example 1-Bench Top Study of Anthocyanins (Ac) Effect on Tumor Necrosis Alpha-Induced Loss of Caco-2 Cell Barrier Integrity

The capacity of anthocyanins (AC) and seven AC-rich extracts containing different types of AC were measured to determine their ability to inhibit tumor necrosis alpha (TNFα) induced permeabilization of Caco-2 cell monolayers. The AC were also tested to determine the relationship between AC chemical structure/conformation and to determine the extent of the AC content to the extent of any protective capacity of the ACs to inhibit TNF-α induced permeability of Caco-2 cell monolayers.

### Materials

Caco-2 cells were obtained from the American Type Culture Collection (Rockville, MA).

Cell culture media and reagents, were from Invitrogen/Life technologies (Grand Island, NY).

HBSS 1X (21-022-CV) was obtained from Corning (Manassas, VA).

Millicell cell culture inserts 12 mm and 30 mm (0.4 μm-pore polyester membranes) (PIHP01250 and PIHP03050, respectively) were obtained from EMD Millipore (Hayward, CA).

Fluorescein isothiocyanate (FITC)-dextran (46944-100MG-F) and tumor necrosis factor-a human (TNFα) (T6674-10UG) were obtained from Sigma Chem. Co. (St. Louis, MO).

Human interferon gamma (IFN-7) (#8901SC) was obtained from Cell Signaling Technology (Danvers, MA).

The pure anthocyanins: delphinidin 3-O-glucoside (myrtillin) (0938), cyanidin 3-O-glucoside (kuromanin) chloride (0915S), and malvidin-3-O-glucoside (oenin) (0911S), were obtained from Extrasynthese (Genay Cedex, France).

The anthocyanin rich powdered extracts were provided by Pharmanex Research (Nu Skin Enterprises) and included: black chokeberry extract powder (35% total AC), black rice extract (20% total AC), wild blueberry extract (5% total AC), bilberry extract (36% total AC), crowberry extract powder (30% total AC), blueberry extract (25% total AC), and red grape extract (minimum 10% total AC).

### Methods

The resolution of chemical structures was performed by molecular mechanics (MM2) according to Allinger using the routines available in ChemBio3D Ultra 11.0.1 (Cambridge Scientific Computing, Inc.). The structure of the anthocyanins appear in FIGS. -5-9.

Caco-2 cells were cultured in a phenol red free minimum essential medium (MEM) at 37° C. and in a 5% (v/vl) CO₂ atmosphere. The MEM media was supplemented with: 10% (v/v) fetal bovine serum; antibiotics (50 μg/ml penicillin, and 50 μg/ml streptomycin); 1% of non-essential amino acid (NEAA); and 1% of sodium pyruvate. The cells were cultured for 21 days after confluence to allow for differentiation into intestinal epithelial cells. Over the course of the 21 days, the media was replaced every 3 days.

After 21 days, the caco-2 cells were differentiated into polarized monolayers in Millicell cell inserts (30 mm, 0.4 μm-pore polyester membranes) and were placed in 6-well plates. The apical chamber consisted of 1.5 ml of media. After an initial addition to the apical chamber of 15 μl of a 1 mg/ml extract solution dissolved in 20% (v/v) ethanol, cells were incubated at 37° C. and 5% (v/vl) CO₂.

Sampling occurred at time zero, 1 hour, and 3 hours. For the initial time-point, 15 μl of medium was withdrawn from the apical chamber and the equivalent volume of extract was added. The plate was gently agitated and a 200 μl sample was immediately withdrawn from the upper chamber. At 1 hour and 3 hours both the apical and basolateral chambers were sampled. All samples were immediately acidified with 2.5 μL of 12M HCl and placed in a −80° C. freezer until time of analysis.

Polyphenolic metabolites present after interacting with the cell layer were identified in each extract by high performance liquid chromatography (HPLC)-mass spectrometry (MS)/MS. The liquid chromatography was performed on an Agilent series 1200 instrument (Agilent Technologies, Santa Clara, CA) coupled with a diode-array detector (DAD) that monitored the cells at wavelengths 280 and 520 nm. A Phenomenex Kinetex F5 pentafluorophenyl HPLC column (2.6 M, 100×4.6 mm) with SecurityGuard® cartridges (PFP, 4.0×2.0 mm) were used for the separation with a flow rate of 0.70 mL min-1 and temperature of 37° C. Injector temperature was set to 4° C. with a 7 μL injection. A binary gradient was employed consisting of 1.0% formic acid (v/v) in water (mobile phase A) and 1.0% formic acid (v/v) in acetonitrile (mobile phase B) (Fisher Scientific, Fair Lawn, NJ). The gradient was as follows: 1% B at 0 minutes, 7.5% B at 7 min, 7.6% B at 14 min, 10% B at 17 min, 12% B at 18.5 min, 30% B at 24 min, 90% B at 25 min, 1% B at 26 to 30 min. Mass spectral data was acquired using an Agilent 6430 triple-quadrupole mass spectrometer with electrospray injection (Agilent Technologies, Santa Clara, CA, USA) and multiple reaction monitoring (MRM) selected as the mode of acquisition. Optimal MS/MS source parameters were set as follows, nebulizer at 40 psi, capillary voltage +4000 V (or −3500 V), gas temperature 325° C. and flow of 5 L-min. Sheath gas was 250° C. and sheath flow of 11 L-min.

Anthocyanin reference standards consisted of malvidin-3-O-glucoside, cyanidine-3-O-glucoside, cyanidine-3-O-galactoside, delphinidin-3-O-glucoside, pelargonidin-3-O-glucoside, and peonidin-3-O-glucoside (Extrasynthese, Genay Cedex, France). Anthocyanins that were detected, but did not have a reference standard quantified by equivalents of malvidin 3-O-glucoside. Phenolic acid reference standards included syringic, vanillic, protocatechuic, 4-hydroxybenzoic, gallic (Sigma-Aldrich St. Louis, MO) and 3-O-methylgallic (Extrasynthese, Genay Cedex, France) acids. Phloroglucinol aldehyde was supplied by Sigma-Aldrich (St. Louis, MO).

To measure transepithelial electrical resistance (TEER) cells were differentiated into polarized monolayers by culture in transwell inserts (12 mm, 0.4 μm-pore polyester membranes) placed in 12-well plates. Epithelial cell monolayers, were initially incubated for 24 hours with interpheron-γ to upregulate the TNF-α receptor. Then the monolayers in the upper chamber were pre-incubated for 30 min with anthocyanin rich extracts (1-10 g/ml) or purified compounds, or with myrtillin chloride, kuromanin chloride, and oenin chloride at 0.25, 0.5, and 1 μM concentration added to the apical compartment. TNFα (5 ng/ml) was subsequently added to the basolateral compartment and cells incubated for 6 more hours.

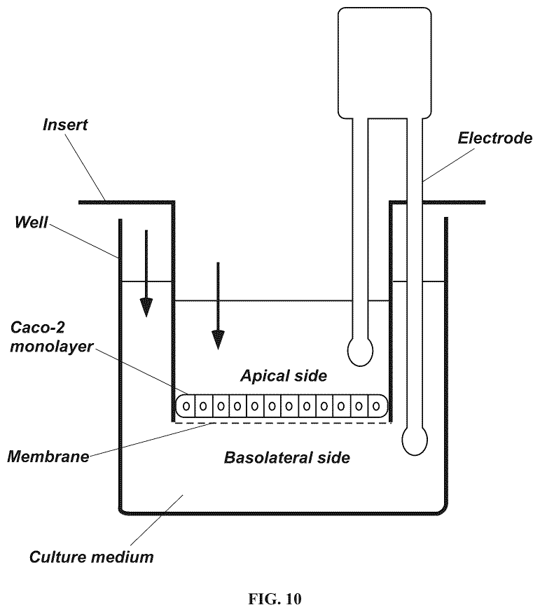

To determine TEER assessment, the incubation media was removed from the apical and basolateral compartments, cells were rinsed with HBSS 1X and the same solution was added to both compartments and TEER was measured. TEER was measured using a Millicell-ERS Resistance System (Millipore, Bedford, MA) that includes a dual electrode volt-ohm-meter. see FIG. 10. TEER was calculated as:

$$T E E R = ( {R}_{m} - {R}_{i} ) \times A . \qquad ( I )$$

wherein, Rₘ, transmembrane resistance; Rᵢ, intrinsic resistance of a cell-free media; and A, the surface area of the membrane in cm²

To determine paracellular transport, apical-to-basolateral clearance of FITC-dextran (4 kDa) was measured. After 6 hours of incubation with TNFα, the media was replaced in both compartments with fresh serum and phenol red-free MEM; FITC-dextran was then added to the apical compartment (100 μM final concentration) and allowed to incubate for 3.5 hours. Subsequently, 100 μl of the medium in the basolateral compartment was collected and diluted with 100 μl of HBSS 1×. The fluorescence was measured at λexc: 485 nm and λem: 530 nm in a fluorescence plate reader. Data was analyzed by one-way analysis of variance (ANOVA) using Statview 5.0 (SAS Institute Inc., Cary, NC). Fisher least significance difference test was used to examine differences between group means. A P value<0.05 was considered statistically significant. Data is shown as mean±SEM.

### Results

The AC concentration in the various extracts as assessed by HPLC-MS/MS is shown in Table 5.

TABLE 5
Anthocyanin Content

| | Black | Black | Wild | | | | |
|---|---|---|---|---|---|---|---|
| | Choke- | Kernel | Blue- | | Crow- | Blue- | Red |
| | berry | Rice | berry | Bilberry | berry | berry | Grape |
| Anthocyanidin | μmol/g extract | | | | | | |
| Cyanidin-3-O-galactoside | 0.87 | | | 3.30 | 10.00 | | 0.75 |
| Cyanidin-3-O-glucoside | 19.29 | 26.50 | | 2.58 | | 3.61 | |
| Cyanidin-3-arabinoside | 9.89 | | | 2.86 | 3.41 | 17.26 | |
| Cyanidin-3-O-rutinoside | | | | | | | |
| Delphinidin-3-O-galactoside | | | | 3.98 | | | |
| Delphinidin-3-O-glucoside | | | 1.25 | 5.16 | 18.59 | 10.85 | 2.16 |
| Delphinidin-3-arabinoside | | | 0.55 | 4.25 | 4.25 | 0.00 | |
| Delphinidin-3-O-rutinoside | | | | | | 26.58 | |
| Petunidin-3-O-galactoside | | | 0.90 | 1.95 | | | |
| Petunidin-3-O-glucoside | | | 1.01 | 3.34 | 9.70 | | 4.69 |
| Petunidin-3-O-arabinoside | | | 0.28 | 1.05 | | | |
| Petunidin-3-(6-acet) glucoside | | | | | | | 1.30 |
| Peonidin-3-O-glucoside | | 2.59 | | 1.67 | 4.53 | | 3.45 |
| Malvidin-3-O-galactoside | | | 1.44 | 3.55 | 2.53 | | 5.07 |
| Malvidin-O-glucoside | | | 1.94 | 1.44 | 25.54 | | 19.96 |
| Malvidin-3-arabinoside | | | 1.12 | 1.12 | 3.45 | | |
| Malvidin-3-(6-acet) glucoside | | | | | | | 1.11 |
| Malvidin-3-(6-acet) galactoside | | | | | | | 4.67 |
| Malvidin-3-(6-coumaryl) | | | | | | | 3.75 |
| glucoside | | | | | | | |
| AC total content | 30.05 | 29.09 | 8.48 | 36.24 | 82.00 | 58.31 | 46.92 |

Among the different extracts, total AC content varied between 8.5 and 82 μmol/g per extract. For the individual ACs, the content ranged between 0 to 30.05; 0 to 37.43; 0 to 9.70; 0 to 3.45; and 0 to 34.56 μmol/g per extract for the different glycosides of cyanidin, delphinidin, petunidin, peonidin and malvidin, respectively.

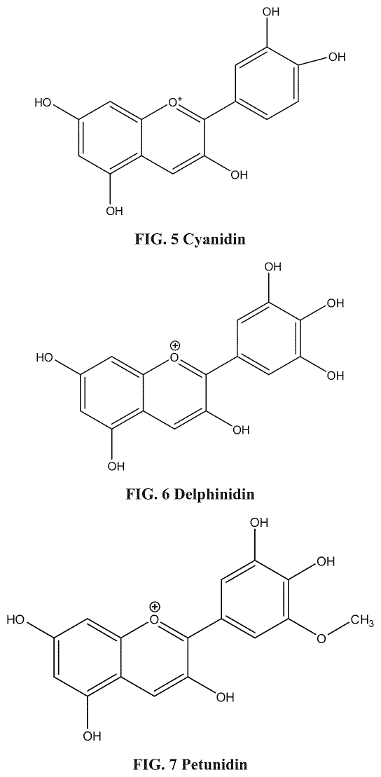

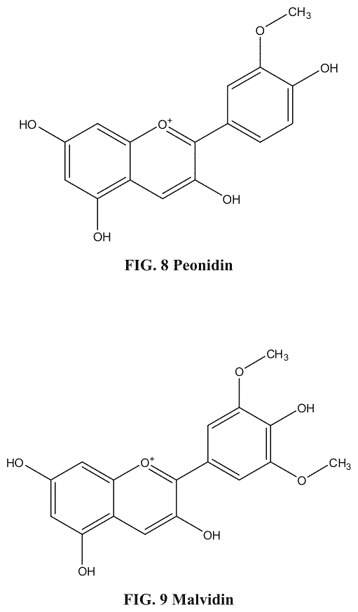

The chemical structure and conformation of the non-glycosylated forms (anthocyanidins) of the AC found in the studied extracts are shown in FIGS. 5-9. While rings A and C are identical for all the cyanidins, the positioning of the B ring with respect to the C ring showed diedric angle values of 39, 37, 34, 39, and 43 degrees for cyanidin, delphinidin, petunidin, peonidin, and malvidin respectively. The position of the B ring may play a role in the beneficial properties of cyanidins and delphinidins. As can be seen in FIGS. 7-9 petunidin, peonidin, and malvidin do not incorporate the same positioning of the B ring.

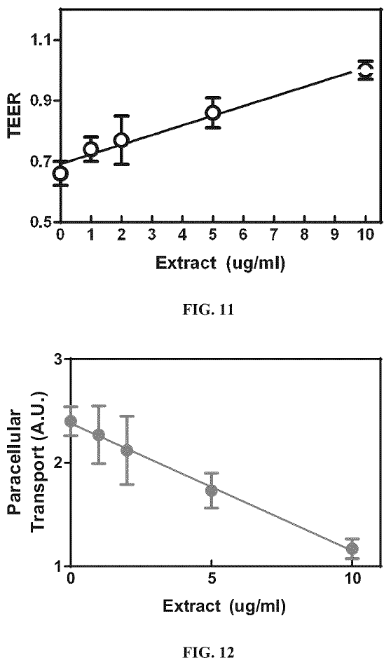

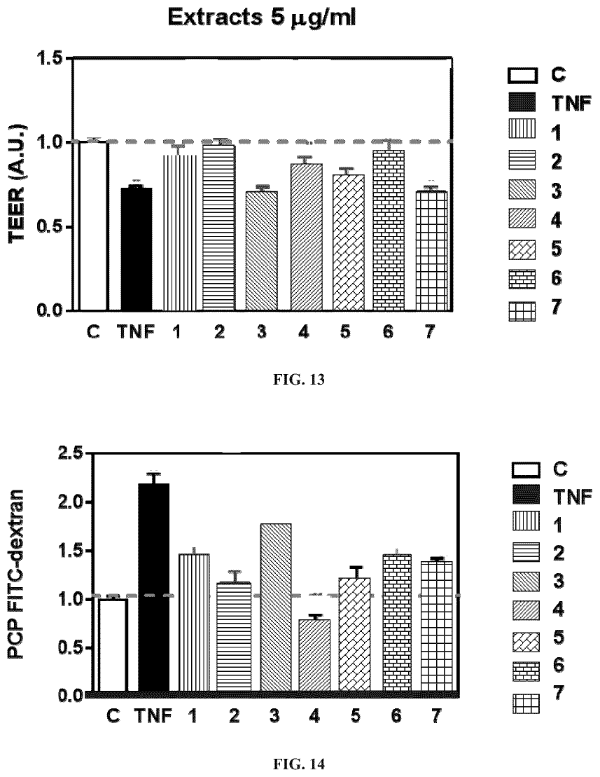

The crowberry extract contained the highest total AC content, 82 mol/g, and delivered one of the larger diversities of individual ACs (cyanidin, delphinidin, petunidin, peonidin and malvidin glycosides)(only bilberry AC were more diverse). Accordingly, the crowberry extract was selected to determine the concentration-dependent capacity of the AC-rich extracts to prevent TNFα-induced permeabilization of Caco-2 monolayers was assessed measuring both TEER and FITC-dextran paracellular transport. Caco-2 monolayers were incubated in the presence of 5 ng/ml TNFα in the lower chamber (basolateral side of the Caco-2 monolayer). This caused, a significant decrease (28%, p<0.05) in TEER and an increase in FITC-dextran paracellular transport (220%, p<0.05). These findings indicate that, under the current experimental conditions, TNFα caused an increase in permeabilization of the Caco-2 cell monolayer. The addition of the crowberry extract (1-10 g/ml) to the upper chamber (apical side of the Caco-2 monolayer) caused a concentration-dependent recovery of the monolayer TEER and inhibition of TNFα-induced FITC-dextran transport to the lower chamber. As can be seen in FIGS. 11 and 12, TEER values increased as the amount of the extract increased and paracellular transport values decreased as the amount of extract increased. This indicated a dose dependent effect. Then the relative capacity of all of the AC-rich extracts to inhibit TNFα-induced Caco-2 cell monolayer permeabilization was determined. At 5 g/ml concentrations, the extracts had differential effects inhibiting TNFα-induced alterations on TEER and FITC-dextran paracellular transport. See FIGS. 13 and 14.

TABLE 6
Extract Indicators

| Indicator | Extract/Formulation |
|---|---|
| C | Control - no extract; TNFα was not induced |
| TNF | Tumor necrosis alpha induced |
| 1 | Black chokeberry extract |
| 2 | Black kernelled rice extract |
| 3 | Wild blueberry |
| 4 | Bilberry extract |
| 5 | Crowberry extract |
| 6 | Blueberry extract |
| 7 | Red grape |

The black chokeberry (1), black kernel rice (2) and blueberry (6) extracts were most effective at inhibiting TNFα-induced alterations on TEER, whereas, the black kernel rice (2), bilberry (4) and crowberry (5) extracts were most effective at inhibiting TNFα-induced alterations on FITC-dextran paracellular transport. The protective actions of the extracts on TNFα-induced monolayer permeabilization was concentration-dependent.

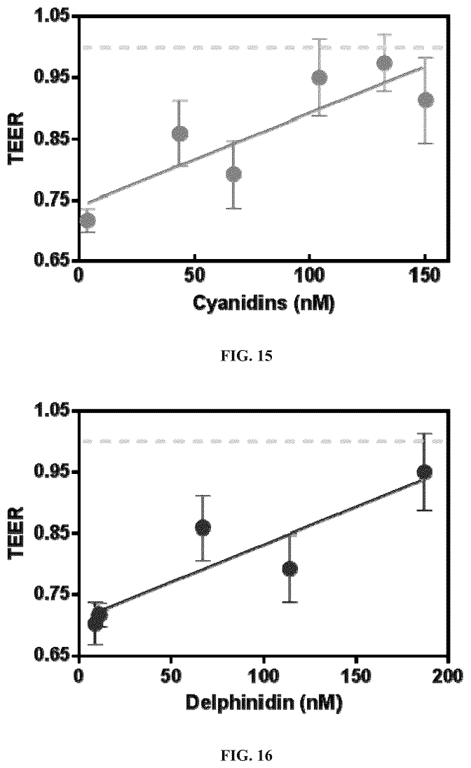

In order to evaluate if the AC present in the extracts could be involved in the beneficial effects of the extracts on Caco-2 cell barrier integrity the correlation between TEER and the total and individual extract AC content was assessed. TEER values were not significantly correlated with the total AC content, or with the content of peonidin, malvidin, and petunidin glycosides in the extracts; however, TEER values were significantly (p<0.03) correlated with the extracts' content of cyanidin (r: 0.73) and delphinidin (r: 0.81) glycosides, see FIGS. 15 and 16, suggesting a protective effect of these specific AC.

TABLE 7
Correlation between anthocyanin content and protective capacity
to prevent TNFα induced TEER decreases

| | R | |
|---|---|---|
| Anthocyanin | (correlation coefficient) | P |
| Total Anthocyanins | 0.100 | 0.48 |
| Cyanidin | 0.730 | 0.03* |
| Delphinidin | 0.810 | 0.03* |
| Peonidin | 0.270 | 0.48 |
| Malvidin | 0.001 | 0.99 |
| Petunidin | 0.140 | 0.48 |

*p < 0.05, only cyanidin and delphinidin concentrations were significantly positively correlated with protective capacity against TNFa induced permeability, in other words, only cyanidins and delphinidins, not total ACs or other individual ACs were protective.

TABLE 8
Epicatechin, Catechin, and Total Catechin Content of the Extracts

| | Extract/ | | | |
|---|---|---|---|---|
| Indicator | Formulation | Epicatechin | Catechin | Total Catechins |
| 1 | Black chokeberry | 8.4 | 4.7 | 13.1 |
| | extract | | | |
| 2 | Black kernelled | — | — | — |
| | rice extract | | | |
| 3 | Wild blueberry | — | 0.9 | 0.9 |
| 4 | Bilberry extract | 2.9 | 2.7 | 5.6 |
| 5 | Crowberry extract | 9.5 | 5.7 | 15.2 |
| 6 | Blueberry extract | — | — | — |
| 7 | Red grape | 2.8 | 3.0 | 5.8 |

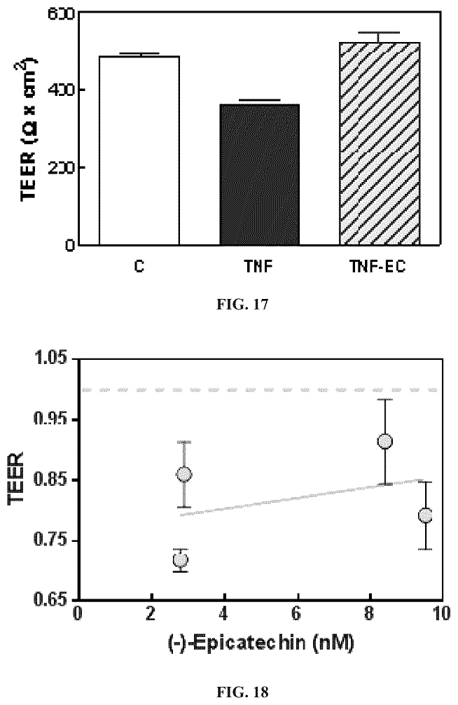

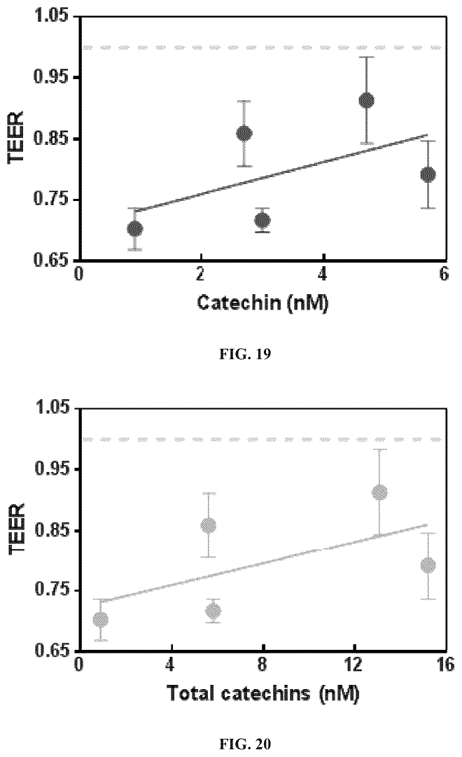

It was discovered that epicatechin and catechins prevents TNFα induced losses of intestinal barrier integrity. See FIGS. 17-20.

### Example 2 —Toxicity Study

A toxicity study was conducted to determine the toxicity of a composition comprising cyanidins, delphinidins and a prebiotic blend after 90 days of oral administration to Wistar rats. The study also evaluated the dose response relationship and the determination of the no-observed adverse effect level.

### Materials-Environment

Healthy Wistar rats (Rattus norvegicus) were selected at random to participate in the study. Then 60 female and 60 male rates between 6-8 weeks old, were allocated to 6 different groups.

Animals were individually housed in standard sized (40.5×24×18.5 cm) polycarbonate cages with a sterilized corn cob bedding. The bedding was changed weekly and more often as necessary to keep the animals clean and dry.

The room temperature was maintained between 22±3C with a relative humidity between 30-70%. Artificial light was cycled for 12 hours of light and 12 hours of night. Air was changed a minimum of 12 times during each hour in the animal room.

Animals were offered a pelleted rodent diet and filtered water ad libitum. The water was placed in polycarbonate bottles with stainless steel sipper tubes.

### Methods

The selected rats were examined by a veterinarian and then were acclimated to the test conditions for 5 days prior to the initial dosing. The rats were allocated into six different groups prior to initiation of the study using computer generated randomization tables. The weight variation of the animals was minimal and did not exceed ±20% of the mean weight.

TABLE 9
Mice and Diet Groupings

| | | | Identification | |
|---|---|---|---|---|
| | | Dose | No. of Animals | |
| Group | Treatment | (mg/kg/day) | Males | Females |
| G1 | Control—distilled water | 0 | 01-10 | 61-70 |
| G2 | Low Dose | 70 | 11-20 | 71-80 |
| G3 | Mild Dose | 350 | 21-30 | 81-90 |
| G4 | High Dose | 700 | 31-40 | 91-100 |
| G5 | Recovery Control | 0 | 41-50 | 101-110 |
| G6 | Recovery High Dose | 700 | 51-60 | 11-120 |

Oral formulations were prepared daily, on the day of dosing. The formulation was dissolved in an aqueous solution. The high dose was anticipated to be equivalent to a human dose.

The formulation consisted of blueberry extract (3.6%), black currant extract (5.2%), black rice extract (15.6%), chicory inulin (48%), and short chain fructooligosaccharides (27.6%) and was administered orally once per day, at the same time each day, for 90 consecutive days. The quantity of the formulation administered was 10 ml/kg of body weight.

All animals were observed daily for clinical signs and symptoms. Animals were observed individually in their home cage, during removal/hand held observations, in an open field, and for sensory reactivity. In the home cage, animals were observed for posture, breathing rate and extent, clonic involuntary movement, tonic involuntary movement, vocalisation, and palperbral closure. During hand held observations animals were observed for palperbral closure, lacrimation, eye and skin examination, piloerection, and salivation. During field observation animals were observed for gait, mobility, arousal, respiration, clonic movement, tonic movement, vocalisation, rearing, urine pools, fecal boluses, stereotype, and bizarre behavior. During sensory reactivity observations, animals were assessed for sensory reactivity including click response, touch response, tail pinch response, and approach response.

The animals were weighed on day 1, weekly during the course of the study, and on the day necropsy.

Urine was collected and analyzed for physical parameters and microscopic examinations on the last week of treatment. In order to collect the urine, animals were housed for 16-18 hours in metabolic cages with graduated tubes attached to the bottom of the cages.

On day 91, blood samples were collected for hematology and chemical analysis before necropsy and animals were fasted overnight for 16-18 hours prior to the blood collection. On day 91, animals in groups 1-4 were necropsied by an overdose of CO₂ and physically examined. The cranial, thoracic, and visceral cavities were opened and examined macroscopically. The organs were trimmed of tissues and fat and weighed. Organs and tissues were then collected and preserved in 10% buffered formalin except for the testes that were fixed in Modified Davidson's fluid. On day 105, animals in groups 5-6 were necropsied, physically examined, and their organs weighed.

### Results

During the study, the physical observations were mostly normal and/or consistent across all of the groups and determined to be toxicology insignificant. No clonic or tonic behaviors were observed. The only bizarre behavior was paper bitting. The paper bitting was observed everyday in all of the groups, including the control groups. The touch response and approach response were fast. The tail pinch was observed as a flinch and the pupil response was normal.

Hematology and clinical chemistry of the treated groups and the control groups did not show any significant differences. Urine chemistry also did not show any significant difference among all the experimental animals and comparable to animals of the G1 and G4 groups.

Mean body weights were similar among the groups in the study. The mean body weights gradually increased over the study. No changes in food consumption were observed. Insignificant changes in organ weight ratio were recorded and considered to be toxicologically insignificant. Histopathological lesions were found in both sexes and in the control groups with random distribution. Accordingly, the lesions were considered to be random in nature.

The study concluded that the formulation did not produce any significant toxological changes in physical, physiological, neurobehavioral, biochemical, hematological, and histopathological parameters in any of the doses administered in the study. None of the treatment related changes were considered to be of toxicological significance.

### Example 3 —Animal Study 1

A mouse model of high fat diet induced obesity was used to investigate the potential capacity of a diet rich in anthocyanins to prevent and/or mitigate obesity induced intestinal inflammation, increased intestinal barrier permeability, and insulin resistance. The effects of anthocyanin supplementation on high fat diet induced alterations in (a) intestinal inflammation, (b) intestinal permeability, (c) gut microbiota, and (d) anthocyanin metabolism were evaluated.

### Materials

Sixty healthy male C57BL/6J mice (20-23 g) were obtained from Jackson laboratories, and housed (4 mice/cage) in standard stainless steel cages. An enrichment environment was provided with the use of mouse houses and bedding. Mice were acclimated for one week before starting the treatments. Mice were grouped and (10 mice/group/time point) fed one of: the control diet; the control diet plus anthocyanins; a high fat diet; a high fat diet plus 2% anthocyanins; a high fat diet plus 20% anthocyanins; or a high fat diet plus 40% anthocyanins. The components in these diets are described below.

The control diet, TD.06416, was obtained from Harlen Teklad, WI. adjusted to approximately 10% calories from fat.

The high fat diet, TD.06414, was obtained from Harlan Teklad, WI. and was a 60% fat diet. This diet was known to induce weight gain and the development of obesity over time. In parallel with weight gain, this diet was known to generate increases in lipid levels (triglycerides, cholesterol, and adipocyte accumulation); increases in blood glucose, the development of insulin insensitivity; and when fed for extended periods of time, diabetes.

The anthocyanin mix was obtained from Nu Skin Enterprises. The mix was comprised of black rice extract, black current extract, and blueberry extract.

### Methods

Mice were housed in stainless steel cages. Four mice were housed in each cage. 10 mice were placed in each group.

TABLE 10
Diet and Dosing Data

| | Dose mg (AC)/ | |
|---|---|---|
| Diet | kg/bodyweight | Calories from fat |
| Control (C) | 0 | 10 wt % |
| C + anthocyanin (AC) | 40 | 10 wt % |
| High Fat (HF) | 0 | 60 wt % |
| HF + 2% AC | 2 | 60 wt % |
| HF + 20% AC | 20 | 60 wt % |
| HF + 40% AC | 40 | 60 wt % |

Each group of mice were fed one of the specialized diets above. Food intake was monitored weekly. Body weight was gathered every two weeks and a fresh diet prepared. Feces were gathered and tested on weeks 0, 2, 4, 6, 8, 10, and 12. Urine was gathered on weeks 4 and 10. Gavage was collected on weeks 8 and 13. Blood was collected on week 10 and midway between weeks 12 and 13. On week 14, the mice were euthanized.

In order to measure intestinal permeabilization, paracellular transport of FITC dextrin was given by gavage at 8 weeks on the diets.

Gene arrays were conducted in different sections of the gastrointestinal tract.

Integrity of the tight junctions were measured as an expression of tight junction proteins and the evaluation of regulatory mechanisms.

Inflammation was determined by measuring F480+ macrophage infiltration in the intestinal mucosa, CRP in plasma, TNF, MCP-1, and iNOS expression in liver/intestinal mucosa.

In addition, ITT/GTTs were performed at weeks 10 and 11 to determine the relationship between insulin sensitivity, intestinal health, and the microbiota.

### Results

During the course of the diet, each group of mice had steady weight gain. The largest amount of weight gain was observed between weeks 0-8 with smaller increases between weeks 8-12. Overall mice on the high fat diet and the high fat diet+2% AC gained the most weight over the study.

TABLE 12
Average Weight Gain

| | Average weight gain (g) | | |
|---|---|---|---|
| Group | Weeks 0-8 | Weeks 8-12 | Total Weight Gain |
| Control (C) | 10.2 | −0.1 | 10.1 |
| C + 40% | 11.9 | 1.6 | 13.5 |
| anthocyanin (AC) | | | |
| High Fat (HF) | 16.43 | 2.87 | 19.3 |
| HF + 2% AC | 15.67 | 1.33 | 17 |
| HF + 20% AC | 15.6 | 2.6 | 18.2 |
| HF + 40% AC | 13.4 | 2.6 | 16 |

The mice fed diets supplemented with 60% of the calories from fat sources, developed obesity, insulin resistance, and intestinal permeabilization within 8-16 weeks of the diet.

Despite the weight gain, the amount of food consumed by mice on the high fat diet was less than the amount of food consumed by mice on the control diets. The amount of food ate by each of the groups ranged between 2-5 grams each week during the study. Animals on the control and the control+anthocyanin diet typically consumed between 3¼ to 4½ grams of food per week. Animals on the high fat diet and high fat+diets typically consumed between 2¼ to 3¼ grams of food per week. Energy intake was similar among the groups. This study solidified the link between diet and overall weight; however, the study also showed that anthocyanin supplementation generally had a lowering trend on overall weight gain.

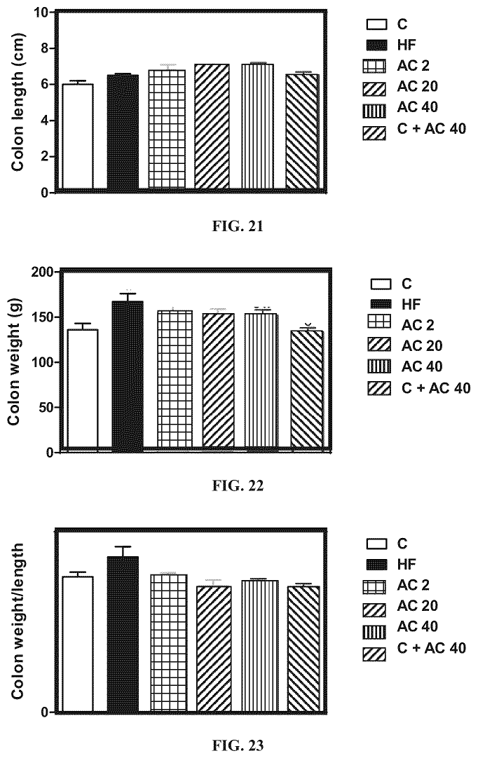

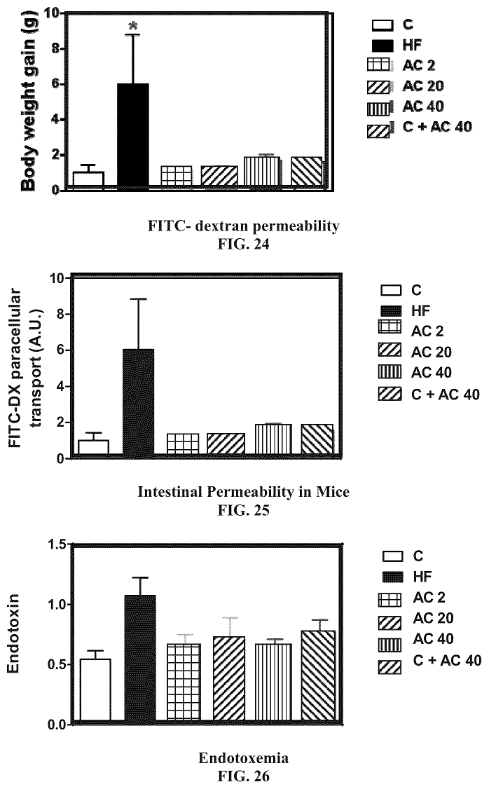

In addition, the overall colon length and weight of the mice was determined. FIGS. 21, 22, and 23. FTC-dextran permeability, intestinal permeability, and endotoxemia were also measured. FIGS. 24, 25, and 26, respectively. Mice with the high fat diet had the most paracellular transport indicating higher intestinal permeability. Supplementing the high fat diet with even small amounts of anthocyanins, decreased or maintained lower levels of intestinal permeability. Mice with a high fat diet also experienced higher amounts of endotoxemia.

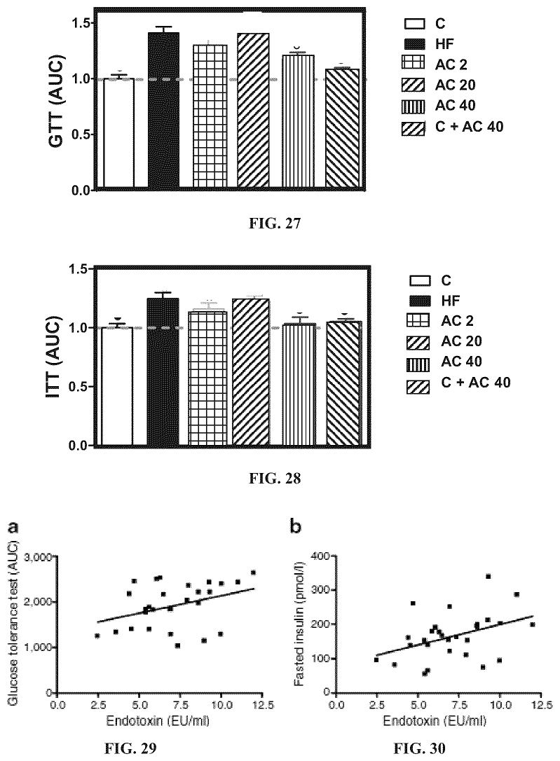

Blood was tested for glucose and insulin concentrations as shown in FIGS. 27 and 28.

TABLE 13
Plasma Glucose and Insulin Concentrations

| | | | HF + | HF + | HF + | C + |
|---|---|---|---|---|---|---|
| | Control | HF | 2% AC | 20% AC | 40% AC | 40% AC |
| Fasting glucose | 164 ± 8 | 230 ± 11 | 206 ± 17 | 225 ± 11 | 161 ± 1.3 | 200 ± 12 |
| (mg/dl) | | | | | | |
| Fasting Insulin | 0.53 ± 0.04 | 1.22 ± 0.11 | 0.88 ± 0.16 | 1.08 ± 0.24 | 0.61 ± 0.10 | 0.47 ± 0.04 |
| Fed Glucose | 124 ± 7 | 173 ± 18 | 150 ± 14 | 178 ± 15 | 136 ± 10 | 150 ± 13 |
| (mg/dl) | | | | | | |
| Fed Insulin | 0.93 ± 0.15 | 1.60 ± 0.24 | 1.56 ± 0.30 | 1.50 ± 0.25 | 0.82 ± 0.17 | 0.99 ± 0.15 |

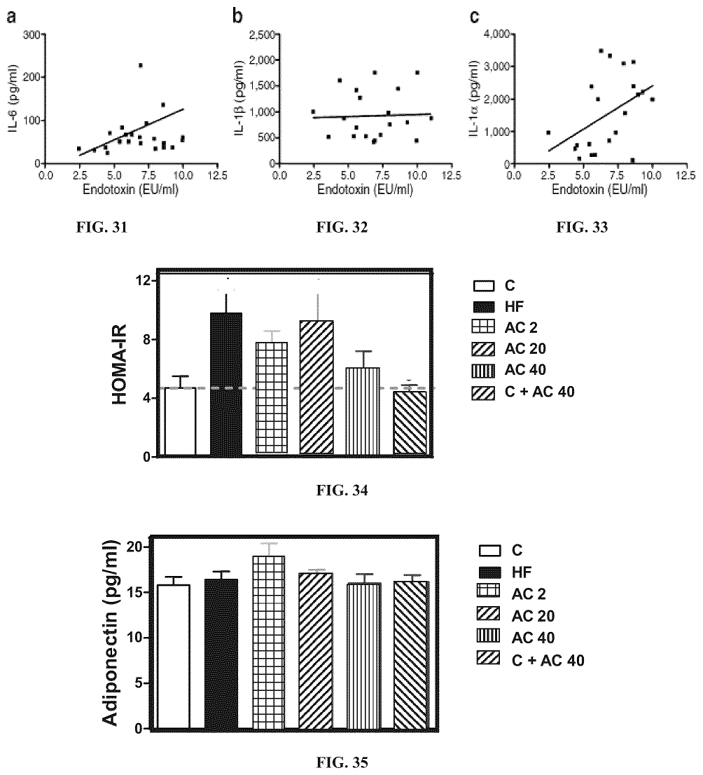

Mice on the high fat diet and on the diet comprising 20% AC exhibited the highest glucose and insulin levels. In addition, as the endotoxins increased so did the glucose tolerance and fasting insulin levels. FIGS. 29 and 30. Endotoxin levels also increased as IL-6 and IL-1α levels increased; however, 1L-0 levels did not show a trend that correlated to endotoxin levels. FIGS. 31-33.

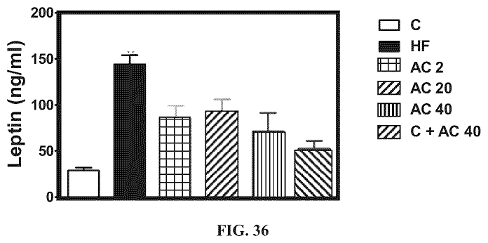

HOMA-IR (homeostasis model assessment of insulin resistance, this is a biomarker of insulin sensitivity), adiponectin, and leptin levels are also shown in FIGS. 34, 35, and 36 respectively. Adiponectin is an adipocyte-derived cytokine with anti-atherogenic, anti-inflammatory, and anti-diabetic properties, which is decreased in obesity. Leptin is another adipocyte-derived cytokine that plays a role in the control of satiety and energy expenditure. Leptin insensitivity, like insulin insensitivity has been associated with weight gain and obesity. Ghrelin is a hormone known to stimulate appetite.

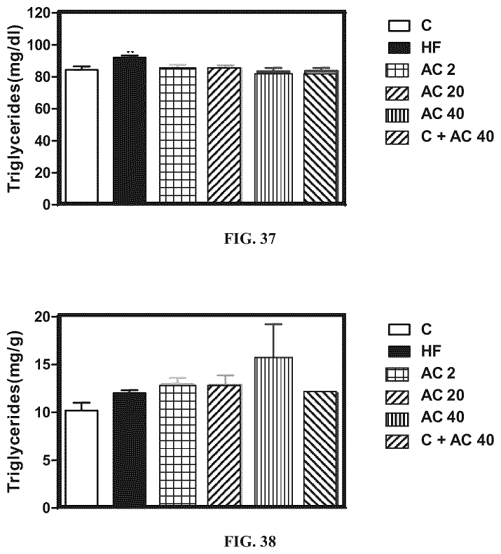

In addition triglyceride and cholesterol levels were measured in the plasma, liver, and the feces. See FIGS. 37-40. As predicted, the high fat diet led to increased plasma triglycerides, however, this increase was prevented by all three AC doses. FIG. 37

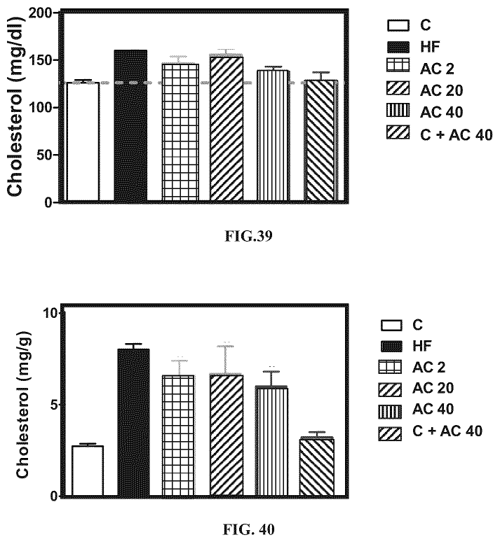

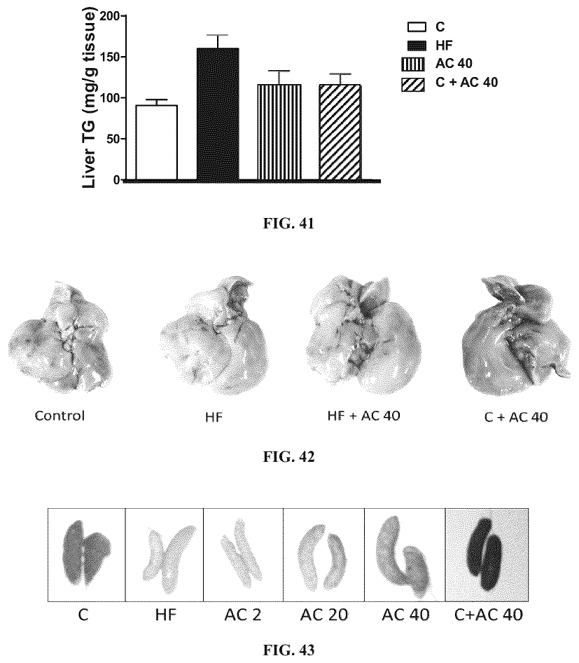

It is well established that a high fat diet leads to hepatic steatosis (fatty liver) in this mouse, therefore, liver triglyceride levels and cholesterol levels were also measured. While triglycerides were significantly increased in the high fat diet control group, the 40% AC diet prevented lipid accumulation in the liver as triglyceride levels in the liver were similar in the control group, control+AC and HF+40% AC groups. FIG. 38. Cholesterol levels were also lower in all of the anthocyanin containing diets than the high fat diet. FIGS. 39 and 40. Liver triglyceride levels were also lower in the anthocyanin containing diets than the high fat diet. FIG. 41. Representative images of mice livers on each diet and their feces can be seen in FIGS. 42 and 43.

Interestingly, the highest triglyceride levels in the feces were found in mice consuming the high fat plus 40% AC diet, suggesting that the anthocyanins inhibited fat absorption, one likely mechanism by which the AC blend may have prevented the high fat diet induced hepatic steatosis and insulin insensitivity. Plasma cholesterol levels were elevated in mice consuming the high fat diet and the high fat+2% and 20% AC groups, only the 40% AC diet attenuated plasma cholesterol levels. This may indicate that a supplement having anthocyanin content can lower plasma total cholesterol levels. All of the high fat diet groups exhibited increased cholesterol levels in the feces, a parameter which did not appear to be influenced by the addition of ACs to the diet.

### Example 4 —Human Study

This study was conducted to determine the effects of a composition containing anthocyanins, inulin, and prebiotic fibers on the microbial composition of obese adults.

### Materials

A single dose of the composition, in Table 14 below, contained 1.9 g inulin, 1.1 g fructooligosaccharides, 144 mg blueberry, 206 mg black currant extract, and 618 mg black rice extract.

TABLE 14
Composition

| | | Anthocyanin |
|---|---|---|
| Active Ingredients | Weight | Content |
| Inulin (Beneo, Orafti GR) | 1,900 mg | N/A |
| Fructooligosaccharides | 1,100 mg | N/A |
| (Ingredion, Nutraflora) | | |
| Blueberry Extract | 144 mg | 35 mg anthocyanins |
| Black Currant Extract | 206 mg | 60 mg anthocyanins |
| Black Rice Extract | 618 mg | 120 mg anthocyanins |

### Methods

An initial screening was conducted two weeks prior to the start of the study. During the initial screening potential participants completed a review of their medical history, indicating all concomitant therapies, and identifying any inclusion and exclusion criteria. Potential participants were also measured for resting blood pressure, heart rate, weight, height, and body mass index. A pregnancy test was performed when applicable.

Fifty-one participants were enrolled in the study after completing the 2-week trial period. The participants were predominately female (73%) and Caucasian (93%). The accepted participants were males and females between 20-60 years of age. They had a BMI ranging from 29.9 to 39.9±1 kg/m²(29.2 to 40.6 kg/m²). Participants agreed to: maintain their level of physical activity throughout the trial period; discontinue the use of pre-biotic and pro-biotics and/or polyphenol supplements; and discontinue eating foods containing anthocyanins (blueberries, blackberries, cherries, grapes, grape juice, pomegranate, raspberries, huckleberries, strawberries, and wine) for two prior to the baseline assessment and during the study.

Participants were instructed to consume one sachet of the formulation every morning with breakfast, by mixing it into their beverage or food of choice. If participants forgot a dose they were instructed to consume the next dose as soon as they remembered. Participants did not consume more than one sachet per day. Participants also recorded diaries for concomitant therapies, adverse events, food records, daily bowel habits, and daily abdominal discomfort. In addition, participants completed bloating and flatulence questioners and had anthropometric measures assessed.

The participants met with the investigational team at the initial assessment and on days 0, 29, and 57 of the study. On day 0 and 57 stool samples, urine samples, and blood samples were collected.

Stool samples were collected within 48 hours of day 0 and day 57. The microbial composition and calprotectin were measured in stool samples by a lab at U of Wisconsin. Microbial composition was measured by 16s rRNA on the illumina platform and R&D Systems (Minneapolis Mn.).

Laboratory parameters (CBC, electrolytes (N, K, Cl, Ca), HbA1c, creatine, AST, ALT, GGT, and bilirubin) were assessed at the initial assessment and at the end of the study.

Urine screenings were administered at the KGK Synergize clinic. Blood parameters were measured by LifeLabs central laboratory by a standard method.

TABLE 15
Tests Conducted Per Visit

| Protocol | Pre Screening | Day 0 | Day 29 | Day 57 |
|---|---|---|---|---|
| Height | X | | | |
| Weight | X | X | X | X |
| Heart Rate | X | | | |
| BP | | X | X | X |
| Medical Review | | X | | X |
| Physical Exam | | X | X | |
| Pregnancy Test | X | | | |
| Fecal Samples | | X | | |
| Formulation Dispensed | | X | | |
| Diaries Dispensed/ | | X | X | X |
| Collected for Daily | | | | |
| Recordings | | | | |
| Bowel Habits Diaries and | | X | | |
| Questionnaires Dispensed | | | | |
| Fecal Sample Kit | | | X | |
| Dispensed | | | | |
| Fecal Sample Kit Collected | | | | X |

Data entry and verification was executed according to KGK Synergize's Standard Operating Procedures. Statistical analysis was conducted on the results of all participants that consumed at least 80% of the treatment doses. Variables were tested for normality and log-normality. Non-normal variables were analyzed by appropriate non-parametric tests. Numerical efficacy and endpoints were formally tested for significance by paired student t-tests.

### Results

Forty-six of the 51 participants originally enrolled in the study completed the study. Four of the participants dropped out after the day 0 visit and before the first comparison measurement on day 29. One individual dropped out between the visits on day 29 and day 57.

### Microbial Diversity

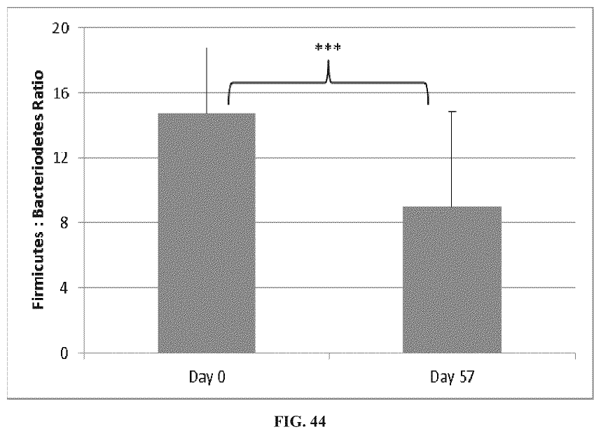

Participants supplementing with the composition experienced a favorable change in their microbial composition as demonstrated by a change in their Firmicutes:Bacteriodetes ratio. FIG. 44. The ratio decreased from 4.98 to 1.45 (Firmicutes decreased 74.9% to 59%; Bacteriodetes increased 13.8% to 34.5%). In addition, Actinobacteria decreased from 8.5% to 3.4%. A total of 8 phyla (6 bacteria, 1 archaea, and 1 other) and 40 genera (7 Actinobacteria, 8 Bacteroidetes, 1 Eurychaeota, 21 Firmicutes, and 3 Proteobacteria) were changed following the supplementation.

TABLE 16
Phylum of Bacteria in Stool

| | | | End of | | |
|---|---|---|---|---|---|
| Kingdom | Phylum | Baseline | Study | P-value | Ratio |
| Other | Other | 2.30E−03 | 3.63E−03 | 0.000 | 0.63 |
| Archaea | Euryarchaeota | 1.12E−04 | 3.40E−05 | 0.010 | 3.29 |
| Bacteria | Other | 6.52E−07 | 6.84E−07 | 1.000 | 0.95 |
| | Acidobacteria | 1.56E−06 | 2.38E−05 | 0.268 | 0.07 |
| | Actinobacteria | 8.54E−02 | 3.37E−02 | 0.000 | 2.54 |
| | Armatimonadetes | 0.00E+00 | 4.46E−07 | 1.000 | — |
| | Bacteroidetes | 1.38E−01 | 3.45E−01 | 0.000 | 0.40 |
| | Caldithrix | 0.00E+00 | 1.15E−06 | 0.371 | — |
| | Chlamydiae | 0.00E+00 | 1.41E−06 | 0.371 | — |
| | Chlorobi | 0.00E+00 | 1.12E−06 | 1.000 | — |
| | Chloroflexi | 1.32E−06 | 2.08E−05 | 0.168 | 0.06 |
| Bacteria | Cyanobacteria | 6.84E−04 | 5.88E−04 | 0.722 | 1.16 |
| | Firmicutes | 7.49E−01 | 5.90E−01 | 0.000 | 1.27 |
| | Fusobacteria | 5.15E−05 | 1.08E−04 | 0.990 | 0.47 |
| | Gemmatimonadetes | 2.61E−07 | 1.89E−05 | 0.059 | 0.01 |
| | Lentisphaerae | 4.28E−06 | 5.80E−05 | 0.017 | 0.07 |
| | NC10 | 2.83E−07 | 6.84E−07 | 0.789 | 0.41 |
| | Nitrospirae | 2.45E−07 | 2.96E−06 | 0.201 | 0.08 |
| | OD1 | 0.00E+00 | 6.70E−07 | 1.000 | — |
| | Planctomycetes | 0.00E+00 | 1.73E−05 | 0.014 | — |
| | Proteobacteria | 1.43E−02 | 2.11E−02 | 0.093 | 0.68 |
| | SBR1093 | 4.65E−07 | 6.77E−06 | 0.295 | 0.07 |
| | Spirochaetes | 5.23E−07 | 6.94E−07 | 1.000 | 0.75 |
| | Synergistetes | 3.65E−05 | 4.24E−05 | 0.636 | 0.86 |
| | TM6 | 0.00E+00 | 1.34E−06 | 1.000 | — |
| | TM7 | 2.44E−04 | 1.03E−04 | 0.000 | 2.38 |
| | Tenericutes | 4.50E−04 | 6.55E−04 | 0.030 | 0.69 |
| | Verrucomicrobia | 9.38E−03 | 4.57E−03 | 0.433 | 2.05 |

TABLE 17
Proportion of Actinobacteria, Bacteroidetes, and Firmicutes in Stool

| | | |
|---|---|---|
| Actinobacteria | Bacteroidetes | Firmicutes |
| Mean ± SD (n) | Mean ± SD (n) | Mean ± SD (n) |
| Median (Min-Max) | Median (Min-Max) | Median (Min-Max) |
| Within Group P Value ^{δ} | Within Group P Value ^{δ} | Within Group P Value ^{δ} |

Proportion of Observed Species

| | | | |
|---|---|---|---|
| Baseline | 0.085 ± 0.068 (45) | 0.138 ± 0.096 (45) | 0.749 ± 0.104 (45) |
| (Day 0) | 0.066 (0.002-0.349) | 0.12 (0.006-0.473) | 0.765 (0.492-0.936) |
| End of Study | 0.034 ± 0.032 (45) | 0.345 ± 0.159 (45) | 0.590 ± 0.141 (45) |
| (Day 57) | 0.028 (0.004-0.15) | 0.341 (0.003-0.639) | 0.59 (0.287-0.89) |
| Change from | −0.052 ± 0.058 (45) | 0.208 ± 0.176 (45) | −0.159 ± 0.170 (45) |
| Baseline to | −0.038 (−0.199-0.029) | 0.227 (−0.254-0.577) | −0.18 (−0.501-0.226) |
| End of Study | p < 0.001* | p < 0.001* | p < 0.001* |

Note:
One participant was unable to provide a fecal sample at the end of study.
Max, maximum;
Min, minimum;
n, number; SD,
standard deviation.
^{δ} Within group comparisons were made using the paired Student t-test.
*Logarithmic transformation required to achieve normality
Probability values P ≤ 0.05 are statistically significant.

### Inflammation

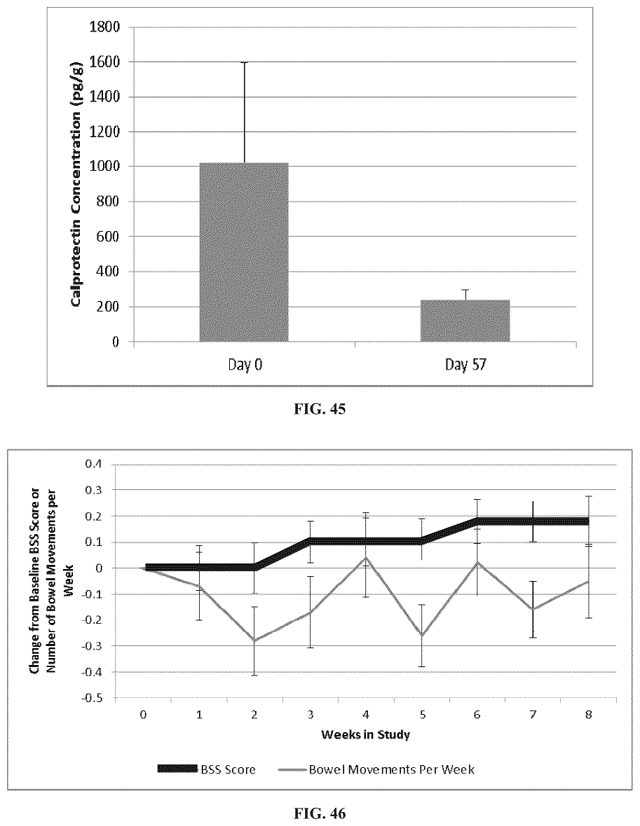

Following the study fecal calprotectin levels were reduced indicating a trend for a reduction in gastrointestinal inflammation. Calprotectin levels are tabulated below and graphically presented in FIG. 45.

TABLE 18
Calprotectin Concentration

| | Calprotectin Concentration (pg/g) |
|---|---|
| | Mean ± SD (n) |
| | Median (Min-Max) |
| | Within Group P Value ^{δ} |
| Baseline | 1,024 ± 3,744 (43) |
| (Day 0) | 113 (7-23,026) |
| End of Study | 240 ± 375 (43) |
| (Day 57) | 106 (5-1,849) |
| Change from | −784 ± 3,727 (43) |
| Baseline to End of Studs | −7 (−22,796-1,474) p = 0.107* |

Note:
One participant was unable to provide a fecal sample at the end of study. Two samples were not available for calprotectin analysis.
g, gram;
Max, maximum;
Min, minimum;
n, number;
pg, picogram;
SD, standard deviation.
^{δ} Within group comparisons were made using the paired Student t-test.
*Logarithmic transformation required to achieve normality.
Probability values P ≤ 0.05 are statistically significant.

### Bowel Habits

A 5% increase was found on weeks 6 and 7 in the Bristol stool score when compared to baseline. The number of bowel movements did not change over the study; however, the bowel habits were improved. FIG. 46. Participants reported a reduction in straining before (33%) and while stopping defecation (51-54%) and a reduction in incomplete bowel movements.

TABLE 19
Change in Daily Bowel Habits

| | | | | |
|---|---|---|---|---|
| | | | Proportion of Bowel | |
| | Number of | | Movements that | Proportion of Bowel |
| | Bowel | Proportion of Bowel | Had | Movements that |
| Bristol Stool Scale | Movements | Movements that Had | Straining to Stop | Were Incomplete |
| (n) | (n/day) | Straining to Start | (%) | (%) |
| Mean ± SD (n) | Mean ± SD (n) | (%) | Mean ± SD (n) | Mean ± SD (n) |
| Median (Min-Max) | Median (Min-Max) | Mean ± SD (n) | Median (Min-Max) | Median (Min-Max) |
| Within Group P | Within Group P | Median (Min-Max) | Within Group P | Within Group P |
| Value ^{δ} | Value ^{δ} | Within Group P Value ^{δ} | Value ^{δ} | Value ^{δ} |

Bowel Habits

| | | | | | |
|---|---|---|---|---|---|
| Basline | 3.88 ± 0.79 (46) | 2.52 ± 1.09 (46) | 17.7 ± 22.8 (46) | 3.9 ± 10.3 (46) | 22.9 ± 27.5 (46) |
| (run-in) | 3.87 (2.17-5.84) | 2 (1-5) | 6.7 (0-95.7) | 0 (0-62.5) | 11.4 (0-91.7) |
| Change from | −0.01 ± 0.59 (46) | −0.07 ± 0.88 (46) | 1.5 ± 20.7 (46) | −1.1 ± 8.2 (46) | −3.8 ± 23.9 (46) |
| Baseline to | 0.04 (−1.4-1.21) | 0 (−2-2) | 0 (−40-45.7) | 0 (−29.2-25) | 0 (−68.3-80) |
| Week 1 | p = 0.924 | p = 0.709* | p = 0.420* | p = 0.185* | p = 0.191* |
| Change from | −0.00 ± 0.65 (46) | −0.28 ± 0.89 (46) | −1.1 ± 17.3 (46) | 0.9 ± 9.6 (46) | −8.7 ± 22.5 (46) |
| Baseline to | −0.03 (−2.2-1.26) | 0 (−2-1) | 0 (−48-36.2) | 0 (−16.7-41.7) | −4 (−79.2-41.7) |
| Week 2 | p = 0.994 | p = 0.066* | p = 0.083* | p = 0.345* | p < 0.001* |
| Change from | 0.10 ± 0.55 (46) | −0.17 ± 0.93 (46) | −2.5 ± 18.0 (46) | 3.5 ± 16.1 (46) | −8.7 ± 24.9 (46) |
| Baseline to | 0.14 (−1.12-1.39) | 0 (−2-2) | 0 (−54-50) | 0 (−10.7-75) | −3.3 (−85-75) |
| Week 3 | p = 0.225 | p = 0.349* | p = 0.089* | p = 0.506* | p < 0.001* |
| Change from | 0.11 ± 0.70 (46) | 0.04 ± 1.03 (46) | −3.7 ± 19.1 (46) | −1.2 ± 6.3 (46) | −10.9 ± 21.9 (46) |
| Baseline to | 0.16 (−1.78-2.47) | 0 (−2-2) | −0.8 (−64.7-36.2) | 0 (−25-25) | −2.6 (−85-23.1) |
| Week 4 | p = 0.309 | p = 0.962* | p = 0.004* | p = 0.025* | p < 0.001* |
| Change from | 0.11 ± 0.54 (46) | −0.26 ± 0.80 (46) | −5.6 ± 18.0 (46) | 0.5 ± 7.5 (46) | −10.7 ± 27.2 (46) |
| Baseline to | 0.17 (−1.52-0.97) | 0 (−2-1) | −3.9 (−64.7-52.4) | 0 (−10.7-37.5) | −4.2 (−85-78.6) |
| Week 5 | p = 0.193 | p = 0.056* | p = 0.002* | p = 0.124* | p < 0.001* |
| Change from | 0.17 ± 0.58 (46) | 0.02 ± 0.86 (46) | −4.8 ± 14.1 (46) | −1.2 ± 5.8 (46) | −11.6 ± 20.6 (46) |
| Basekline to | 0.24 (−1.31-1.56) | 0 (−2-3) | −4 (−40-25) | 0 (−25-14.3) | −4.5 (−85-20) |
| Week 6 | p = 0.051 | p = 0.891* | p < 0.001* | p = 0.167* | p < 0.001* |
| Change from | 0.18 ± 0.53 (46) | −0.15 ± 0.73 (46) | −5.0 ± 16.9 (46) | −2.0 ± 7.4 (46) | −11.6 ± 24.2 (46) |
| Baseline to | 0.22 (−1.29-1.42) | 0 (−1-2) | 0 (−54.7-52.4) | 0 (−33.9-14.3) | −6.7 (−91.7-37.2) |
| Week 7 | p = 0.028 | p = 0.284* | p = 0.002* | p = 0.046* | p < 0.001* |
| Change from | 0.18 ± 0.64 (46) | −0.07 ± 0.95 (46) | −5.6 ± 19.1 (46) | −2.2 ± 8.3 (46) | −27 ± 108 (46) |
| Baseline to | 0.28 (−1.39-1.71) | 0 (−2-2) | −1.7 (−47.6-50) | 0 (−25-33.3) | −6 (−721-50) |
| Week 8 | p = 0.063 | p = 0.432* | p < 0.001* | p = 0.019* | p < 0.001* |

Max, maximum;
Min, minimum;
n, number;
%, percent;
SD, standard deviation.
^{δ} Within group comparisons were made using the paired Student t-test.
*Logarithmic transformation required to achieve normality
Probability values P ≤ 0.05 are statistically significant.

### Bloating, Discomfort, and Gas.

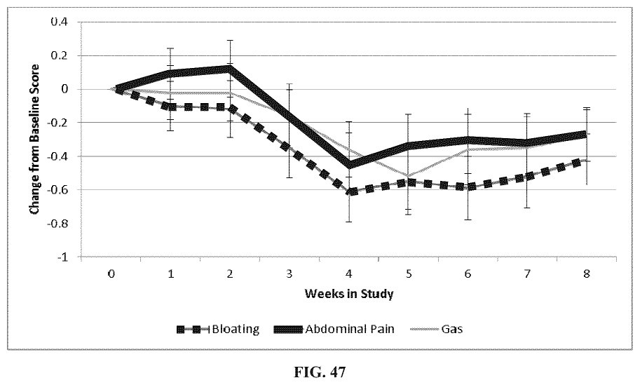

Participants experienced a reduction in bloating from weeks 3 through 8 when compared to baseline (week 4, 41%; week 5, 52%; and week 8, 50%). FIG. 47.

TABLE 20
Bloating

| | | |
|---|---|---|
| | Proportion of Days | Proportion of Days |
| | With Bloating | With Bloating |
| Bloating Severity | Due to Food | Due to Menstrual Cycle |
| (n) | (%) | (%; Females Only) |
| Mean ± SD (n) | Mean ± SD (n) | Mean ± SD (n) |
| Median (Min-Max) | Median (Min-Max) | Median (Min-Max) |
| Within Group P Value ^{δ} | Within Group P Value ^{δ} | Within Group P Value ^{δ} |

Bloating Results

| | | | |
|---|---|---|---|
| Baseline | 2.11 ± 2.07 (46) | 26 ± 33 (46) | 6.3 ± 10.9 (34) |
| (Day 0) | 1.93 (0-9.07) | 7 (0-100) | 0 (0-35.7) |
| Change from | −0.10 ± 1.00 (46) | −7.8 ± 23.6 (46) | −2.1 ± 20.2 (34) |
| Baseline to Week 1 | −0.14 (−2.57-2.29) | 0 (−64.3-42.9) | 0 (−35.7-92.9) |
| | p = 0.125λ | p = 0.034‡ | p = 0.125‡ |
| Change from | −0.12 ± 1.15 (46) | −7 ± 31 (46) | −1.3 ± 22.5 (34) |
| Baseline to Week 2 | −0.14 (−2.36-3.07) | 0 (−93-64) | 0 (−35.7-92.9) |
| | p = 0.054λ | p = 0.167‡ | p = 0.223‡ |
| Change from | −0.35 ± 1.21 (46) | −8.7 ± 27.2 (46) | −3.4 ± 13.9 (34) |
| Baseline to Week 3 | −0.24 (−3.71-2.14) | −7.1 (−85.7-57.1) | 0 (−35.7-42.9) |
| | p = 0.005λ | p = 0.056‡ | p = 0.145‡ |
| Change from | −0.61 ± 1.21 (46) | −10.6 ± 27.5 (46) | −4.2 ± 13.6 (34) |
| Baseline to Week 4 | −0.21 (−3.71-1.43) | −7.1 (−85.7-42.9) | 0 (−35.7-28.6) |
| | p < 0.001λ | p = 0.024‡ | p = 0.106‡ |
| Change from | −0.55 ± 1.34 (46) | −13.4 ± 28.9 (46) | −0.4 ± 21.1 (34) |
| Baseline to Week 5 | −0.14 (−3.71-3.07) | −7.1 (−85.7-42.9) | 0 (−35.7-85.7) |
| | p = 0.001λ | p = 0.005‡ | p = 0.462‡ |
| Change from | −0.59 ± 1.25 (46) | −12 ± 33 (46) | −2.5 ± 18.1 (34) |
| Baseline to Week 6 | −0.14 (−3.86-1.71) | 0 (−100-43) | 0 (−35.7-64.3) |
| | p < 0.001λ | p = 0.052‡ | p = 0.326‡ |
| Change from | −0.52 ± 1.24 (46) | −10 ± 32 (46) | −2.1 ± 19.3 (34) |
| Baseline to Week 7 | −0.14 (−3.5-1.93) | −7 (−100-43) | 0 (−35.7-92.9) |
| | p = 0.002λ | p = 0.101‡ | p = 0.082‡ |
| Change from | −0.42 ± 1.01 (46) | −13 ± 38 (46) | −2.9 ± 20.1 (34) |
| Baseline to Week 8 | −0.11 (−2.79-1.57) | −7 (−100-79) | 0 (−35.7-92.9) |
| | p < 0.001λ | p = 0.015‡ | p = 0.082‡ |

Max, maximum;
Min, minimum;
n, number;
%, percent;
SD, standard deviation.
^{δ} Within group comparisons were made using the paired Student t-test.
‡Within group comparisons were made using the signed-rank test.
λSquare root transformation required to achieve normality
Probability values P ≤ 0.05 are statistically significant.

Participants also experienced a reduction in abdominal pain and gas. Gas severity decreased overtime from 22% at week 5 to 11% at week 8. FIG. 47.

TABLE 21
Change in Abdominal Pain

| | | |
|---|---|---|
| | Proportion of Days | Proportion of Days |
| Abdominal Pain | With Abdominal Pain | With Abdominal Pain |
| Severity | Due to Food | Due to Menstrual Cycle |
| (n) | (%) | (%; Females Only) |
| Mean ± SD (n) | Mean ± SD (n) | Mean ± SD (n) |
| Median (Min-Max) | Median (Min-Max) | Median (Min-Max) |
| Within Group P Value ^{δ} | Within Group P Value ^{δ} | Within Group P Value ^{δ} |

Abdominal Pain Results

| | | | |
|---|---|---|---|
| Baseline | 1.90 ± 1.83 (46) | 23.3 ± 29.6 (46) | 5.5 ± 10.3 (34) |
| (Day 0) | 1.64 (0-8.43) | 10.7 (0-100) | 0 (0-35.7) |
| Change from | 0.09 ± 1.03 (46) | −6.8 ± 20.3 (46) | −0.8 ± 19.9 (34) |
| Baseline to Week 1 | 0 (−1.95-3.07) | 0 (−57.1-50) | 0 (−35.7-92.9) |
| | p = 0.914* | p = 0.025‡ | p = 0.287‡ |
| Change from | 0.12 ± 1.15 (46) | −4.0 ± 26.3 (46) | 0.0 ± 23.2 (34) |
| Baseline to Week 2 | 0 (−1.86-3.07) | 0 (−85.7-64.3) | 0 (−35.7-92.9) |
| | p = 0.972* | p = 0.185‡ | p = 0.305‡ |
| Change from | −0.18 ± 1.18 (46) | −8.1 ± 20.1 (46) | −1.7 ± 15.0 (34) |
| Baseline to Week 3 | −0.07 (−3.14-3.71) | −7.1 (−71.4-28.6) | 0 (−35.7-42.9) |
| | p = 0.050* | p = 0.011‡ | p = 0.448‡ |
| Change from | −0.44 ± 1.22 (46) | −9.0 ± 23.3 (46) | −1.7 ± 17.0 (34) |
| Baseline to Week 4 | −0.14 (−4.57-2.57) | −7.1 (−78.6-42.9) | 0 (−35.7-50) |
| | p = 0.006* | p = 0.017‡ | p = 0.555‡ |
| Change from | −0.35 ± 1.35 (46) | −11.5 ± 24.8 (46) | 1.3 ± 20.2 (34) |
| Baseline to Week 5 | −0.14 (−3.57-3.64) | −7.1 (−85.7-42.9) | 0 (−35.7-85.7) |
| | p = 0.037* | p = 0.002‡ | p = 0.723‡ |
| Change from | −0.32 ± 1.30 (46) | −9.9 ± 28.9 (46) | −1.3 ± 20.6 (34) |
| Baseline to Week 6 | −0.11 (−2.71-4.71) | −3.6 (−100-42.9) | 0 (−35.7-92.9) |
| | p = 0.031* | p = 0.040‡ | p = 0.288‡ |
| Change from | −0.33 ± 1.16 (46) | −9.3 ± 26.5 (46) | −0.4 ± 19.4 (34) |
| Baseline to Week 7 | −0.04 (−3.43-3.29) | −7.1 (−100-42.9) | 0 (−35.7-92.9) |
| | p = 0.014* | p = 0.021‡ | p = 0.304‡ |
| Change from | −0.28 ± 0.98 (46) | −9 ± 34 (46) | −2.1 ± 19.9 (34) |
| Baseline to Week 8 | 0 (−2.57-2) | −7 (−100-79) | 0 (−35.7-92.9) |
| | p = 0.014* | p = 0.051‡ | p = 0.118‡ |

Max, maximum;
Min, minimum;
n, number;
%, percent;
SD, standard deviation.
^{δ} Within group comparisons were made using the paired Student t-test.
‡Within group comparisons were made using the signed-rank test.
λ Square root transformation required to achieve normality
Probability values P ≤ 0.05 are statistically significant.

TABLE 22
Change in Gas

| | | |
|---|---|---|
| | | Proportion of Days |
| | Proportion of Days | With Gas |
| Gas Severity | With Gas | Due to Menstrual Cycle |
| (n) | Due to Food (%) | (%; Females Only) |
| Mean ± SD (n) | Mean ± SD (n) | Mean ± SD (n) |
| Median (Min-Max) | Median (Min-Max) | Median (Min-Max) |
| Within Group P Value ^{δ} | Within Group P Value ^{δ} | Within Group P Value ^{δ} |

Gas Results

| | | | |
|---|---|---|---|
| Baseline | 2.40 ± 1.96 (46) | 36 ± 36 (46) | 5.3 ± 10.3 (34) |
| | 2.04 (0-8.5) | 18 (0-100) | 0 (0-35.7) |
| Change from | −0.02 ± 1.09 (46) | −10.7 ± 29.6 (46) | −1.5 ± 19.5 (34) |
| Baseline to Week 1 | 0.07 (−2.57-3.36) | −7.1 (−71.4-92.9) | 0 (−35.7-92.9) |
| | p = 0.843* | p = 0.003‡ | p = 0.136‡ |
| Change from | −0.02 ± 1.16 (46) | −10 ± 36 (46) | −1.5 ± 18.2 (34) |
| Baseline to Week 2 | −0.11 (−2.71-1.93) | −7 (−86-79) | 0 (−35.7-78.6) |
| | p = 0.462* | p = 0.031‡ | p = 0.210‡ |
| Change from | −0.16 ± 1.29 (46) | −12.6 ± 27.3 (46) | −2.3 ± 13.6 (34) |
| Baseline to Week 3 | −0.25 (−2.5-4.57) | −7.1 (−85.7-50) | 0 (−35.7-42.9) |
| | p = 0.340* | p = 0.004‡ | p = 0.325‡ |
| Change from | −0.36 ± 1.11 (46) | −9 ± 35 (46) | −3.2 ± 14.3 (34) |
| Baseline to Week 4 | −0.21 (−4-2.36) | −7 (−86-93) | 0 (−35.7-42.9) |
| | p = 0.062* | p = 0.118‡ | p = 0.156‡ |
| Change from | −0.52 ± 1.31 (46) | −17.2 ± 29.8 (46) | 1.1 ± 20.0 (34) |
| Baseline to Week 5 | −0.29 (−4.14-2.07) | −7.1 (−85.7-35.7) | 0 (−35.7-85.7) |
| | p = 0.007* | p < 0.001‡ | p = 0.608‡ |
| Change from | −0.35 ± 1.38 (46) | −16 ± 32 (46) | −1.5 ± 17.8 (34) |
| Baseline to Week 6 | −0.36 (−4-2.86) | −7 (−100-36) | 0 (−35.7-64.3) |
| | p = 0.056* | p = 0.007‡ | p = 0.408‡ |
| Change from | −0.34 ± 1.22 (46) | −14 ± 34 (46) | −1.5 ± 16.9 (34) |
| Baseline to Week 7 | −0.25 (−3.71-2.5) | −7 (−100-43) | 0 (−35.7-78.6) |
| | p = 0.043* | p = 0.020‡ | p = 0.166‡ |
| Change from | −0.26 ± 1.07 (46) | −16 ± 39 (46) | −1.5 ± 20.4 (34) |
| Baseline to Week 8 | −0.21 (−2.57-2.5) | −7 (−100-79) | 0 (−35.7-92.9) |
| | p = 0.035* | p = 0.006‡ | p = 0.212‡ |

Max, maximum;
Min, minimum;
n, number;
%, percent;
SD, standard deviation.
^{δ} Within group comparisons were made using the paired Student t-test.
‡Within group comparisons were made using the signed-rank test.
λ Square root transformation required to achieve normality
Probability values P ≤ 0.05 are statistically significant.

### Vital Signs

Participants experienced a clinically insignificant reduction in diastolic blood pressure from baseline to the end of the study.

TABLE 23
Vital Signs

| | | | | |
|---|---|---|---|---|
| Systolic Blood | Diastolic Blood | Heart Rate | | |
| Pressure (mmHg) | Pressure (mmHg) | (BPM) | Weight (kg) | BMI (kg/m²) |
| Mean ± SD (n) | Mean ± SD (n) | Mean ± SD (n) | Mean ± SD (n) | Mean ± SD (n) |
| Median (Min-Max) | Median (Min-Max) | Median (Min-Max) | Median (Min-Max) | Median (Min-Max) |
| Within Group P | Within Group P | Within Group P | Within Group P | Within Group P |
| Value ^{δ} | Value ^{δ} | Value ^{δ} | Value ^{δ} | Value ^{δ} |

Vitals and Anthropometric Measures

| | | | | | |
|---|---|---|---|---|---|
| Screening | 119.8 ± 11.8 (51) | 76.5 ± 10.1 (51) | 74.1 ± 12.0 (51) | 95.7 ± 14.2 (51) | 34.2 ± 3.2 (51) |
| | 119.3 (92.3-146) | 75 (57-98.7) | 75 (55-109) | 95 (71.6-137.1) | 33.7 (29.2-40.6) |
| Baseline | 120.0 ± 12.0 (51) | 79.4 ± 9.2 (51) | 72.9 ± 10.4 (51) | 95.2 ± 14.0 (51) | 34.1 ± 3.1 (51) |
| (Day 0) | 121.7 (95.7-143) | 80.3 (58-101.7) | 74 (46-91) | 94 (71.4-134.4) | 33.6 (28.7-40.8) |
| Mid-point | 120.5 ± 12.5 (50) | 77.3 ± 8.7 (50) | 74.0 ± 10.0 (50) | 94.7 ± 12.9 (50) | 34.1 ± 3.1 (50) |
| (Day 29) | 122.3 (92.3-151) | 77.8 (56-102) | 72.5 (52-100) | 94.8 (71.6-128.3) | 33.9 (28.5-40.8) |
| End of | 120.3 ± 11.1 (47) | 76.7 ± 8.3 (47) | 71.6 ± 10.2 (47) | 94.4 ± 13.1 (47) | 33.9 ± 3.2 (47) |
| Study | 120 (98.7-141) | 75.7 (60.3-96.3) | 70 (49-97) | 94 (71-127.4) | 33.5 (28.7-41) |
| (Day 57) | | | | | |
| Change | 0.7 ± 11.6 (50) | −1.9 ± 8.6 (50) | 1.2 ± 9.3 (50) | 0.36 ± 1.38 (50) | 0.14 ± 0.51 (50) |
| from | 0.5 (−28.7-35) | −0.8 (−23.7-16.7) | 1 (−19-34) | 0.25 (−2.3-4.6) | 0.11 (−0.95-1.8) |
| Baseline to | p = 0.663 | p = 0.122 | p = 0.365 | p = 0.071 | p = 0.065 |
| Mid-point | | | | | |
| Change | 0.4 ± 10.0 (47) | −2.9 ± 8.2 (47) | −0.9 ± 8.7 (47) | 0.06 ± 2.02 (47) | 0.04 ± 0.73 (47) |
| from | 2.3 (−25.7-22.7) | −3.7 (−18.7-17.7) | −1 (−26-20) | 0.1 (−5.2-5.4) | 0.04 (−1.72 −2.11) |
| Baseline to | p = 0.799 | p = 0.020 | p = 0.506 | p = 0.847 | p = 0.716 |
| End of | | | | | |
| Study | | | | | |

BPM, beats per minute;
kg, kilograms;
Max, maximum;
mmHg, m, meter;
millimeters of mercury;
Min, minimum;
N, number;
SD, standard deviation.
^{δ} Within group comparisons were made using the paired Student t-test.
Probability values P ≤ 0.05 are statistically significant

### CONCLUSION

Overall the study indicated that the formulation was capable of improving the intestinal microbiome in mildly to moderately obese yet otherwise healthy individuals. Over the study, participants experienced a significant reduction in the Firmicutes and a significant increase in Bacteroidetes proportions. The Firmicutes:Bacteroidetes ratio decreased from day 0 to day 57 with supplementation. The ratio changed from 4.98 to 1.45. In addition, Actinobacteria levels decreased. These three phyla make up about 97% of the bacterial composition at baseline (97.2%) and following supplementation (96.9%).

Thirty adverse events were recorded by 18 participants in the study. Of the thirty reported incidents, eleven were assessed as unlikely and 7 were assessed as unrelated. Two of the adverse events were determined to be likely and included feces discoloration and tooth discoloration. The ten possible adverse events included reports of abdominal discomfort, diarrhea, feces discoloration, frequent bowel movements, and vomiting.

Formulations, methods for the production of these formulations, and uses for the formulations have been described. It will be readily apparent to those skilled in the art, however various changes and modifications of an obvious nature may be made without departing from the spirit of the invention, and all such changes and modifications are considered to fall within the scope of the invention as defined by the appended claims. Such changes and modifications would include, but not be limited to, the incipient ingredients added to affect the capsule, tablet, powder, lotion, food, powder, or bar manufacturing process as well as vitamins, flavorings, and carriers. Other such changes or modifications would include the use of herbs or other botanical products containing the combinations of the preferred embodiments disclosed above. Many additional modifications and variations of the embodiments described herein may be made without departing from the scope, as is apparent to those skilled in the art. The specific embodiments described herein are offered by way of example only.

1. An intestinal health promoting composition, comprising a combination of cyanidins and delphinidins, in an amount sufficient to treat intestinal hyperpermeability.

2. The composition of claim 1, wherein the cyanidins and the delphinidins are collectively present in an amount that maintains intestinal permeability.

3. The composition of claim 1, wherein the cyanidins and the delphinidins are collectively present in an amount that reduces intestinal hyperpermeability.

4. The composition of claim 1, wherein a source of at least one of the cyanidins and the delphinidins are derived from a black rice component, a blueberry component, a black current component, a crowberry component, a bilberry component, black chokeberry component, or a combination thereof.

5. The composition of claim 4, wherein the source of the cyanidins and the delphinidins are derived from the black rice component, the blueberry component, and the black current component.

6. The composition of claim 4, wherein the composition comprises the black rice component and the black rice component is derived from a member selected from the group consisting of black rice kernels, black rice concentrate, black rice extract, black rice powder, or a combination thereof.

7-15. (canceled)

16. The composition of claim 4, wherein the composition comprise the blueberry component and the blueberry component comprises a member selected from the group consisting of blueberry fruit, blueberry extract, blueberry concentrate, blueberry juice, blueberry powder, or a combination thereof.

17-25. (canceled)

26. The composition of claim 4, wherein the composition comprises the black current component and the black current component comprises a member selected from the group consisting of black current fruit, black current extract, black current concentrate, black current juice, black current powder, or a combination thereof.

27-32. (canceled)

33. The composition of claim 4, wherein the composition comprises the crowberry component and the crowberry component comprises a member selected from the group consisting of crowberry fruit, crowberry extract, crowberry concentrate, crowberry juice, crowberry powder, or a combination thereof.

34-40. (canceled)

41. The composition of claim 4, wherein the composition comprises the bilberry component and the bilberry component comprises a member selected from the group consisting of bilberry fruit, bilberry extract, bilberry concentrate, bilberry juice, bilberry powder, or a combination thereof.

42-48. (canceled)

49. The composition of claim 4, wherein the source of at least one of the cyanidins and the delphinidins is derived from a black rice component, a blueberry component, and a black current component.

50-51. (canceled)

52. The composition of claim 1, further comprising a prebiotic blend.

53-66. (canceled)

67. The composition of claim 1, further comprising a prebiotic blend of inulin and fructooligosacharides.

68-69. (canceled)

70. The composition of claim 1, further comprising a pharmaceutically acceptable carrier.

71. The composition of claim 1, further comprising a sweetener, a preservative, a flavoring, a thickener, or a combination thereof.

72. The composition of claim 1, wherein the composition is an oral dosage form.

73-96. (canceled)

97. A method of treating a condition or disorder related to gastrointestinal health in a subject comprising maximizing tight junction integrity in epithelial cells of gastrointestinal tract of the subject.

98. The method of claim 97, wherein the gastrointestinal health of the subject is improved.

99-104. (canceled)

105. The method of claim 97, wherein the condition or disorder comprises inflammation, inflammatory bowel disease, irritable bowel syndrome, chronic intestinal diseases, celiac disease, Crohn's disease, ulcerative colitis, food intolerances, dyspepsia, low levels of chronic intestinal inflammation, gastrointestinal infections, or a combination thereof.

106-108. (canceled)

109. The method of claim 97, wherein the condition or disorder comprises insufficient absorption of nutrients, endotoxemia, intestinal hyperpermeability, or a combination thereof.

110. The method of claim 97, wherein the condition or disorder comprises obesity, obesity associated pathologies, allergies, cardiovascular conditions, type I diabetes, type II diabetes, rheumatoid arthritis, insulin resistance, cancer, metabolic syndrome, asthma, neurodegenerative diseases, or a combination thereof.

111. The method of claim 97, wherein the condition or disorder is a cardiovascular condition.

112-115. (canceled)

116. The method of claim 97, wherein the condition is peripheral insulin resistance and the method reduces peripheral insulin resistance.

117. The method of claim 97, wherein condition or disorder is type I diabetes or type II diabetes.

118. The method of claim 97, wherein the condition or disorder comprises a nitric oxide related disorder, expression of iNOS, expression of COX-2, NADPH oxidase, or a combination thereof.

119. The method of claim 97, wherein the condition or disorder stems from pathogens, antigens, and pro-inflammatory factors that pass through the tight junctions in the epithelial cells of the gastrointestinal tract.

120. The method of claim 97, wherein maximizing tight junction integrity comprises protecting the gastrointestinal tract of the subject from TNFα induced permealization of a monolayer of the epithelial cells.

121. (canceled)

122. The method of claim 97, wherein the method further comprises increasing transepithelial electrical resistance in the epithelial cells.

123. The method of claim 97, wherein the method further comprises increasing FITC dextran paracellular transport.

124. The method of claim 97, wherein the condition stems from pro-inflammatory factors and the pro-inflammatory factors comprise advanced glycation end products.

125. The method of claim 97, wherein the condition stems from pro-inflammatory factors and the pro-inflammatory factors comprise lipopolysaccharides.

126. The method of claim 97, wherein the condition stems from pro-inflammatory factors and the pro-inflammatory factors comprise cytokines tumor necrosis alpha (TNF-α), IL-6, or a combination thereof.

127. The method of claim 97, wherein the condition or disorder relates to conditions or disorders associated with signaling pathways NF-kB, ERK1/2, or a combination thereof.

128. The method of claim 97, wherein the maximizing of the tight junction integrity mitigates high fat induced intestinal permeabilization.

129. The method of claim 97, wherein the epithelial cells comprise Caco-2 cell monolayers.

130. The method of claim 97, wherein the method further comprises optimizing a balance of gut microbiota in the gastrointestinal tract.

131-146. (canceled)

147. The method of claim 97, wherein the method comprises providing a fuel source for commensal bacteria.
